(19) United States
(12) Patent Application Publication
(10) Pub. No.: US 2022/0175218 A1
(43) Pub. Date: Jun. 9, 2022

(54) INFORMATION PROCESSING DEVICE, ENDOSCOPE CONTROL DEVICE, INFORMATION PROCESSING METHOD AND OPERATING METHOD OF ENDOSCOPE CONTROL DEVICE

(71) Applicant: OLYMPUS CORPORATION, Tokyo (JP)

(72) Inventor: Hirokazu NISHIMURA, Tokyo (JP)

(73) Assignee: OLYMPUS CORPORATION, Tokyo (JP)

(21) Appl. No.: 17/677,354

(22) Filed: Feb. 22, 2022

Related U.S. Application Data
(63) Continuation of application No. PCT/JP2019/034269, filed on Aug. 30, 2019.

(51) Int. Cl. A61B 1/005; A61B 1/00

(52) U.S. Cl. CPC A61B 1/009; A61B 1/00006; A61B 1/0051; A61B 1/000096

(57) ABSTRACT

An information processing device includes a processor including one or more hardware components. The processor is configured to obtain a classification result that a kind of an insertion shape of an endoscope insertion portion inserted into a subject is classified as one of a plurality of predetermined kinds, and output the classification result.

### CROSS REFERENCE TO RELATED APPLICATION

This application is a continuation application of PCT/JP2019/034269 filed on Aug. 30, 2019, the entire contents of which are incorporated herein by this reference.

### BACKGROUND OF THE INVENTION

### 1. Field of the Invention

The present invention relates to an information processing device, an endoscope control device, an information processing method, and an operating method of the endoscope control device.

### 2. Description of the Related Art

In endoscope observation, an insertion operation for inserting an elongated insertion portion having flexibility into a deep part in a subject is performed. Technologies for supporting the insertion operation of the insertion portion have been conventionally proposed in the endoscope field.

Specifically, for example, Japanese Patent No. 4274854 discloses an endoscope insertion shape analysis device configured to analyze an insertion shape of an endoscope insertion portion inserted into a body cavity. When a loop is formed by an insertion operation of the endoscope insertion portion, an operation method is displayed for disentangling the loop and linearizing the endoscope insertion portion.

Recently in the field of endoscope, discussions have been made on technologies for automating an insertion operation of the insertion portion.

### SUMMARY OF THE INVENTION

An information processing device according to an aspect of the present invention is an information processing device configured to classify a kind of an insertion shape of an endoscope insertion portion by using information related to the insertion shape of the endoscope insertion portion inserted into a subject. The information processing device includes a processor including one or more hardware components. The processor is configured to obtain a classification result that the kind of the insertion shape of the endoscope insertion portion inserted into the subject is classified as one of a plurality of predetermined kinds, and output the classification result.

An endoscope control device according to an aspect of the present invention is an endoscope control device configured to perform control of an insertion operation of an endoscope insertion portion by using information related to an insertion shape of the endoscope insertion portion inserted into a subject. The endoscope control device includes a processor including one or more hardware components. The processor is configured to obtain an extraction result by extracting one or more constituent elements of the insertion shape of the endoscope insertion portion inserted into the subject, and perform control of the insertion operation of the endoscope insertion portion based on the extraction result.

An information processing method according to an aspect of the present invention includes: obtaining a classification result that a kind of an insertion shape of an endoscope insertion portion inserted into a subject is classified as one of a plurality of predetermined kinds; and outputting the classification result.

An operating method of an endoscope control device according to an aspect of the present invention is an operating method of an endoscope control device configured to perform control of an insertion operation of an endoscope insertion portion by using information related to an insertion shape of the endoscope insertion portion inserted into a subject, the method including: performing processing for obtaining an extraction result by extracting one or more constituent elements of the insertion shape of the endoscope insertion portion inserted into the subject; and performing control of the insertion operation of the endoscope insertion portion based on the extraction result.

### BRIEF DESCRIPTION OF THE DRAWINGS

FIG. 1 is a diagram illustrating a configuration of a main part of an endoscope system including an endoscope control device according to a first embodiment of the present invention;

FIG. 2 is a block diagram for description of a specific configuration of the endoscope system according to the first embodiment;

FIG. 3 is a diagram illustrating an example of an insertion shape image generated in the endoscope system according to the first embodiment;

FIG. 4A is a diagram illustrating an example of an insertion shape image generated in the endoscope system according to the first embodiment;

FIG. 4B is a diagram illustrating an example of an insertion shape image generated in the endoscope system according to the first embodiment;

FIG. 5A is a diagram illustrating an example of an insertion shape image generated in the endoscope system according to the first embodiment;

FIG. 5B is a diagram illustrating an example of an insertion shape image generated in the endoscope system according to the first embodiment;

FIG. 6A is a diagram illustrating an example of an insertion shape image generated in the endoscope system according to the first embodiment;

FIG. 6B is a diagram illustrating an example of an insertion shape image generated in the endoscope system according to the first embodiment;

FIG. 7A is a diagram illustrating an example of an insertion shape image generated in the endoscope system according to the first embodiment;

FIG. 7B is a diagram illustrating an example of an insertion shape image generated in the endoscope system according to the first embodiment;

FIG. 8A is a diagram illustrating an example of an insertion shape image generated in the endoscope system according to the first embodiment;

FIG. 8B is a diagram illustrating an example of an insertion shape image generated in the endoscope system according to the first embodiment;

FIG. 9A is a diagram illustrating an example of an insertion shape image generated in the endoscope system according to the first embodiment;

FIG. 9B is a diagram illustrating an example of an insertion shape image generated in the endoscope system according to the first embodiment;

FIG. 10 is a diagram illustrating an example of an insertion shape image generated in the endoscope system according to the first embodiment;

FIG. 11A is a diagram illustrating an example of an insertion shape image generated in the endoscope system according to the first embodiment;

FIG. 11B is a diagram illustrating an example of an insertion shape image generated in the endoscope system according to the first embodiment;

FIG. 12A is a diagram illustrating an example of an insertion shape image generated in the endoscope system according to the first embodiment;

FIG. 12B is a diagram illustrating an example of an insertion shape image generated in the endoscope system according to the first embodiment;

FIG. 13A is a diagram illustrating an example in which temporal transition of a kind of an insertion shape of an insertion portion is visualized by using information recorded in the endoscope system according to the first embodiment;

FIG. 13B is a diagram illustrating an example in which temporal transition of the kind of the insertion shape of the insertion portion is visualized by using information recorded in the endoscope system according to the first embodiment;

FIG. 13C is a diagram illustrating an example in which temporal transition of the kind of the insertion shape of the insertion portion is visualized by using information recorded in the endoscope system according to the first embodiment;

FIG. 14 is a flowchart for description of an outline of control performed in an endoscope system according to a modification of the first embodiment;

FIG. 15A is a diagram illustrating an example of an endoscope image generated in the endoscope system according to the embodiment;

FIG. 15B is a diagram illustrating an example of a processing result image obtained when processing for detecting a position of a lumen region is performed on the endoscope image in FIG. 15A;

FIG. 15C is a diagram for description of control performed when the processing result image in FIG. 15B is obtained;

FIG. 16 is a block diagram for description of a specific configuration of an endoscope system according to a second embodiment;

FIG. 17A is a diagram illustrating an example of an image illustrating an extraction result obtained when constituent elements of the insertion shape of the insertion portion are extracted from an insertion shape image generated in the endoscope system according to the second embodiment;

FIG. 17B is a diagram illustrating an example of an image illustrating an extraction result obtained when constituent elements of the insertion shape of the insertion portion are extracted from an insertion shape image generated in the endoscope system according to the second embodiment;

FIG. 17C is a diagram illustrating an example of an image illustrating an extraction result obtained when constituent elements of the insertion shape of the insertion portion are extracted from an insertion shape image generated in the endoscope system according to the second embodiment; and

FIG. 17D is a diagram illustrating an example of an image illustrating an extraction result obtained when constituent elements of the insertion shape of the insertion portion are extracted from an insertion shape image generated in the endoscope system according to the second embodiment.

### DETAILED DESCRIPTION OF THE PREFERRED EMBODIMENTS

Embodiments of the present invention will be described below with reference to the accompanying drawings.

### First Embodiment

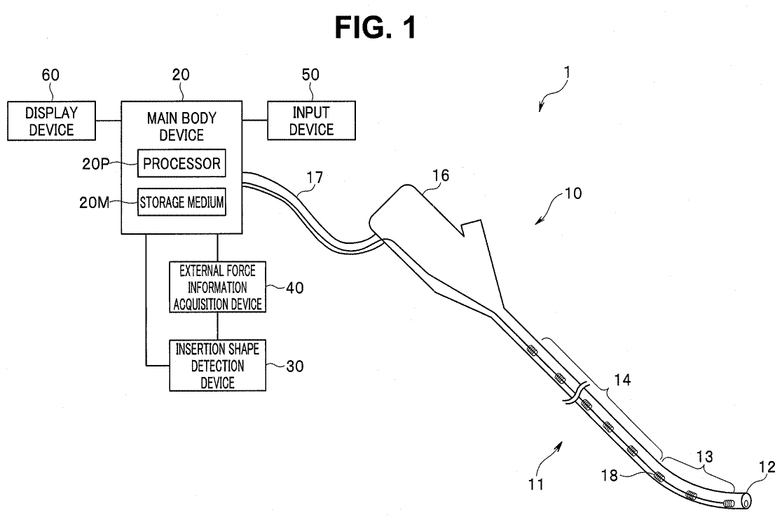

FIGS. 1 to 15C relate to a first embodiment.

For example, as illustrated in FIG. 1, an endoscope system 1 includes an endoscope 10, a main body device 20, an insertion shape detection device 30, an external force information acquisition device 40, an input device 50, and a display device 60. FIG. 1 is a diagram illustrating the configuration of a main part of the endoscope system including an endoscope control device (information processing device) according to an embodiment.

The endoscope 10 includes an insertion portion 11 inserted into a subject, an operation portion 16 provided on a base end side of the insertion portion 11, and a universal cord 17 extended from the operation portion 16. The endoscope 10 is configured to be removably connected to the main body device 20 through a scope connector (not illustrated) provided at an end part of the universal cord 17.

Note that a light guide (not illustrated) for transmitting illumination light supplied from the main body device 20 is provided inside the insertion portion 11, the operation portion 16, and the universal cord 17 described above.

The insertion portion 11 has flexibility and an elongated shape. The insertion portion 11 includes, sequentially from a distal end side, a hard distal end portion 12, a bending portion 13 formed to be bendable, and an elongated flexible tube portion 14.

A plurality of source coils 18 configured to generate a magnetic field in accordance with a coil drive signal supplied from the main body device 20 are disposed at a predetermined interval in a longitudinal direction of the insertion portion 11 inside the distal end portion 12, the bending portion 13, and the flexible tube portion 14.

The distal end portion 12 is provided with an illumination window (not illustrated) for emitting, to an object, illumination light transmitted through the light guide provided inside the insertion portion 11. The distal end portion 12 is also provided with an image pickup unit 110 (not illustrated in FIG. 1) configured to perform operation in accordance with an image pickup control signal supplied from the main body device 20, perform image pickup of the object illuminated with the illumination light emitted through the illumination window, and output an image pickup signal.

The bending portion 13 is configured to be able to bend in accordance with control by a bending control unit 242 to be described later. The bending portion 13 is configured to be able to bend in accordance with an operation of an angle knob (not illustrated) provided at the operation portion 16.

The operation portion 16 has a shape with which the operation portion 16 can be grasped and operated by a user such as a surgeon. The operation portion 16 is provided with an angle knob configured to be operated for bending the bending portion 13 in four directions of up, down, right, and left directions intersecting with a longitudinal axis of the insertion portion 11. The operation portion 16 is also provided with one or more scope switches (not illustrated) through which an instruction can be provided in accordance with an input operation by the user.

As illustrated in FIG. 1, the main body device 20 includes a processor 20P including one or more hardware components, and a storage medium 20M. The main body device 20 is configured to be removably connected to the endoscope 10 through the universal cord 17.

The main body device 20 is configured to be removably connected to components of the insertion shape detection device 30, the input device 50, and the display device 60. The main body device 20 is configured to perform operation in accordance with an instruction from the input device 50. The main body device 20 is configured to generate an endoscope image based on an image pickup signal outputted from the endoscope 10 and perform operation for displaying the generated endoscope image on the display device 60.

In the present embodiment, the main body device 20 is configured to generate and output various kinds of control signals for controlling operation of the endoscope 10. The main body device 20 has functions as the endoscope control device and is configured to perform control of an insertion operation of the insertion portion 11 by using insertion shape information (to be described later) outputted from the insertion shape detection device 30.

In addition, the main body device 20 is configured to perform operation for generating an insertion shape image in accordance with the insertion shape information outputted from the insertion shape detection device 30 and displaying the generated insertion shape image on the display device 60.

The insertion shape detection device 30 is configured to detect a magnetic field generated from each of the source coils 18 provided at the insertion portion 11 and acquire a position of each of the plurality of source coils 18 based on intensity of the detected magnetic field. In addition, the insertion shape detection device 30 is configured to generate insertion shape information indicating the position of each of the plurality of source coils 18, which is acquired as described above, and output the insertion shape information to the main body device 20 and the external force information acquisition device 40.

Specifically, the insertion shape detection device 30 is configured to acquire insertion shape information by detecting an insertion shape of the insertion portion inserted into the subject and output the acquired insertion shape information to the main body device 20 and the external force information acquisition device 40.

The external force information acquisition device 40 stores, for example, data of curvatures (or curvature radii) and bending angles at a plurality of predetermined positions on the insertion portion 11 in a state in which no external force is applied, and data of curvatures (or curvature radii) and bending angles at the plurality of predetermined positions, which are acquired in a state in which predetermined external force is applied in every expected direction at any position on the insertion portion 11.

In the present embodiment, for example, the external force information acquisition device 40 is configured to specify the position of each of the plurality of source coils 18 provided in the insertion portion 11 based on insertion shape information outputted from the insertion shape detection device 30, and acquire a magnitude and a direction of external force at the position of each of the plurality of source coils 18 by referring to various kinds of data stored in advance based on a curvature (or curvature radius) and a bending angle at the position of each of the plurality of source coils 18.

In addition, the external force information acquisition device 40 is configured to generate external force information indicating the magnitude and direction of external force at the position of each of the plurality of source coils 18, which is acquired as described above, and output the external force information to the main body device 20.

Note that, in the present embodiment, a method disclosed in Japanese Patent No. 5851204 or a method disclosed in Japanese Patent No. 5897092 may be used as a method by which the external force information acquisition device 40 calculates external force at the position of each of the plurality of source coils 18 provided in the insertion portion 11.

In the present embodiment, when an electronic component such as a distortion sensor, a pressure sensor, an acceleration sensor, a gyro sensor, or a wireless element is provided in the insertion portion 11, the external force information acquisition device 40 may be configured to calculate external force at the position of each of the plurality of source coils 18 based on a signal outputted from the electronic component.

The input device 50 includes one or more input interfaces operated by the user such as a mouse, a keyboard, and a touch panel. The input device 50 is configured to be able to output an instruction in accordance with an operation by the user to the main body device 20.

The display device 60 includes a liquid crystal monitor or the like. The display device 60 is configured to be able to display an endoscope image outputted from the main body device 20 and the like on a screen.

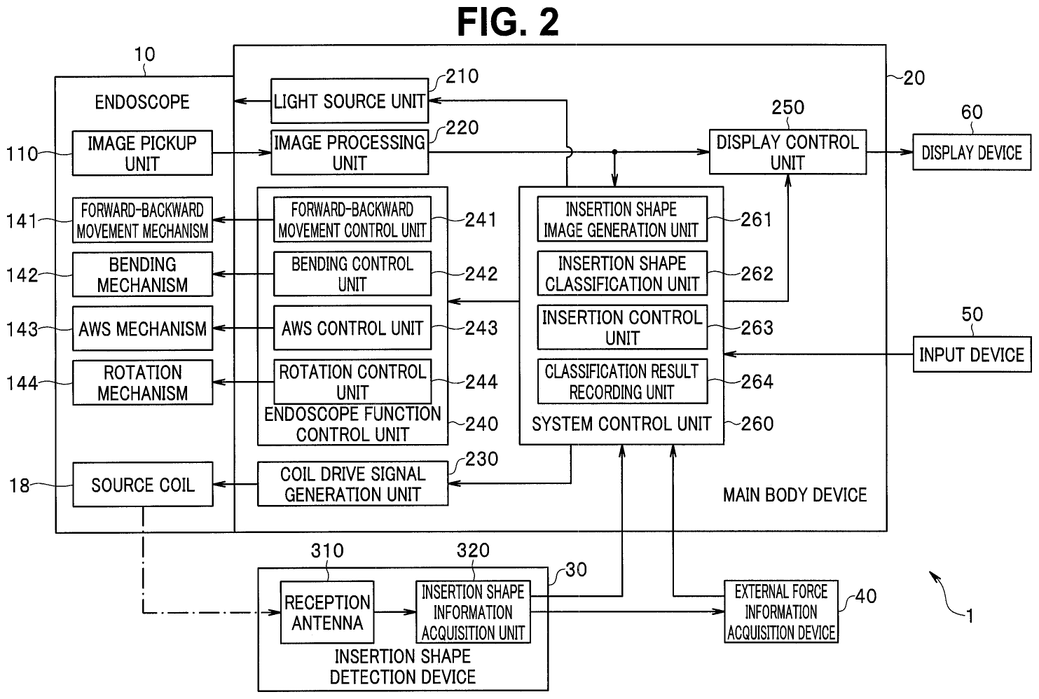

Subsequently, a specific configuration of the endoscope system including the endoscope control device of the first embodiment will be described with reference to FIG. 2.

FIG. 2 is a block diagram for description of a specific configuration of the endoscope system according to the first embodiment.

As illustrated in FIG. 2, the endoscope 10 includes the source coils 18, the image pickup unit 110, a forward-backward movement mechanism 141, a bending mechanism 142, an AWS mechanism 143, and a rotation mechanism 144. FIG. 2 is a block diagram for description of the specific configuration of the endoscope system according to the first embodiment.

The image pickup unit 110 includes, for example, an observation window on which return light from an object illuminated with illumination light is incident, and an image sensor such as a color CCD configured to perform image pickup of the return light and output an image pickup signal.

The forward-backward movement mechanism 141 includes, for example, a pair of rollers disposed at facing positions on both sides of the insertion portion 11, and a motor configured to supply rotational drive force for rotating the pair of rollers. For example, the forward-backward movement mechanism 141 is configured to drive the motor in accordance with a forward-backward movement control signal outputted from the main body device 20 and rotate the pair of rollers in accordance with the rotational drive force supplied from the motor, thereby selectively performing any one of operation for moving forward the insertion portion 11 and operation for moving backward the insertion portion 11.

The bending mechanism 142 includes, for example, a plurality of bending pieces provided in the bending portion 13, a plurality of wires coupled with the plurality of bending pieces, and a motor configured to supply rotational drive force for pulling the plurality of wires. For example, the bending mechanism 142 is configured to drive the motor in accordance with a bending control signal outputted from the main body device 20 and change a pulling amount of each of the plurality of wires in accordance with the rotational drive force supplied from the motor, thereby bending the bending portion 13 in four directions of up, down, right, and left directions.

The AWS (air feeding, water feeding, and suction) mechanism 143 includes, for example, two pipelines of an air-water feeding pipeline and a suction pipeline provided inside the endoscope 10 (the insertion portion 11, the operation portion 16, and the universal cord 17), and an electromagnetic valve configured to perform operation to open one of the two pipelines and close the other pipeline.

In the present embodiment, for example, when operation for opening the air-water feeding pipeline is performed at the electromagnetic valve in accordance with an AWS control signal outputted from the main body device 20, the AWS mechanism 143 is configured to be able to cause fluid including at least one of water or air supplied from the main body device 20 to circulate through the air-water feeding pipeline and discharge through a discharge port formed at the distal end portion 12.

In addition, for example, when operation for opening the suction pipeline is performed at the electromagnetic valve in accordance with an AWS control signal outputted from the main body device 20, the AWS mechanism 143 is configured to be able to apply suction force generated at the main body device 20 to the suction pipeline and suck, with the suction force, an object existing near a suction port formed at the distal end portion 12.

The rotation mechanism 144 includes, for example, a grasping member configured to grasp the insertion portion 11 on the base end side of the flexible tube portion 14, and a motor configured to supply rotational drive force for rotating the grasping member. For example, the rotation mechanism 144 is configured to drive the motor in accordance with a rotation control signal outputted from the main body device 20 and rotate the grasping member in accordance with the rotational drive force supplied from the motor, thereby rotating the insertion portion 11 about an insertion axis (longitudinal axis).

<Details of Main Body Device 20>

As illustrated in FIG. 2, the main body device 20 includes a light source unit 210, an image processing unit 220, a coil drive signal generation unit 230, an endoscope function control unit 240, a display control unit 250, and a system control unit 260.

The light source unit 210 includes, for example, one or more LEDs or one or more lamps as light sources. The light source unit 210 is configured to be able to generate illumination light for illuminating inside of the subject into which the insertion portion 11 is inserted, and supply the illumination light to the endoscope 10. In addition, the light source unit 210 is configured to be able to change light quantity of illumination light in accordance with a system control signal supplied from the system control unit 260.

The image processing unit 220 includes, for example, an image processing circuit. The image processing unit 220 is configured to generate an endoscope image by providing predetermined processing to an image pickup signal outputted from the endoscope 10, and output the generated endoscope image to the display control unit 250 and the system control unit 260.

The coil drive signal generation unit 230 includes, for example, a drive circuit. The coil drive signal generation unit 230 is configured to generate and output a coil drive signal for driving the source coils 18 in accordance with a system control signal supplied from the system control unit 260.

The endoscope function control unit 240 is configured to perform, based on an insertion control signal supplied from the system control unit 260, operation for controlling a function achieved by the endoscope 10. Specifically, the endoscope function control unit 240 is configured to perform operation for controlling at least one of a forward-backward movement function achieved by the forward-backward movement mechanism 141, a bending function achieved by the bending mechanism 142, an AWS function achieved by the AWS mechanism 143, or a rotation function achieved by the rotation mechanism 144. The endoscope function control unit 240 includes a forward-backward movement control unit 241, the bending control unit 242, an AWS control unit 243, and a rotation control unit 244.

The forward-backward movement control unit 241 is configured to generate and output, based on an insertion control signal supplied from the system control unit 260, a forward-backward movement control signal for controlling operation of the forward-backward movement mechanism 141. Specifically, the forward-backward movement control unit 241 is configured to generate and output, based on an insertion control signal supplied from the system control unit 260, for example, a forward-backward movement control signal for controlling a rotational state of the motor provided in the forward-backward movement mechanism 141.

The bending control unit 242 is configured to generate and output, based on an insertion control signal supplied from the system control unit 260, a bending control signal for controlling operation of the bending mechanism 142. Specifically, the bending control unit 242 is configured to generate and output, based on an insertion control signal supplied from the system control unit 260, for example, a bending control signal for controlling a rotational state of the motor provided in the bending mechanism 142.

The AWS control unit 243 is configured to be able to selectively perform any one of operation for supplying fluid including at least one of water or air to the endoscope 10 and operation for generating suction force for sucking an object existing near the suction port of the distal end portion 12, by controlling a non-illustrated pump or the like based on an insertion control signal supplied from the system control unit 260.

The AWS control unit 243 is also configured to generate and output an AWS control signal for controlling operation of the AWS mechanism 143. Specifically, the AWS control unit 243 is configured to generate and output, based on an insertion control signal supplied from the system control unit 260, for example, an AWS control signal for controlling an operation state of the electromagnetic valve provided in the AWS mechanism 143.

The rotation control unit 244 is configured to generate and output, based on an insertion control signal supplied from the system control unit 260, a rotation control signal for controlling operation of the rotation mechanism 144. Specifically, the rotation control unit 244 is configured to generate and output, based on an insertion control signal supplied from the system control unit 260, for example, a rotation control signal for controlling a rotational state of the motor provided in the rotation mechanism 144.

In other words, the endoscope function control unit 240 is configured to be able to generate and output, based on insertion control signals supplied from the system control unit 260, as control signals corresponding to basic operations achieved by functions of the endoscope 10, control signals corresponding to a pushing operation corresponding to an operation for moving forward the insertion portion 11, a pulling operation corresponding to an operation for moving backward the insertion portion 11, an angle operation corresponding to an operation for bending the bending portion 13 to align an orientation of the distal end portion 12 with a direction (for example, one of eight directions) intersecting with the insertion axis (longitudinal axis) of the insertion portion 11, a twisting operation corresponding to an operation for rotating the insertion portion 11 about the insertion axis (longitudinal axis), an air feeding operation for ejecting gas toward a front side of the distal end portion 12, a water feeding operation for ejecting liquid toward the front side of the distal end portion 12, and a suction operation for sucking a tissue or the like on the front side of the distal end portion 12.

The display control unit 250 performs processing for generating a display image including an endoscope image outputted from the image processing unit 220 and performs processing for displaying the generated display image on the display device 60. The display control unit 250 also performs processing for displaying, on the display device 60, an insertion shape image (to be described later) outputted from the system control unit 260.

The system control unit 260 generates and outputs system control signals for performing operation in accordance with instructions and the like from the operation portion 16 and the input device 50. The system control unit 260 includes an insertion shape image generation unit 261, an insertion shape classification unit 262, an insertion control unit 263, and a classification result recording unit 264.

The insertion shape image generation unit 261 generates, based on insertion shape information (to be described later) outputted from the insertion shape detection device 30, an insertion shape image two-dimensionally illustrating the insertion shape of the insertion portion 11 inserted into the subject. The insertion shape image generation unit 261 outputs the insertion shape image generated as described above to the display control unit 250.

The insertion shape classification unit 262 performs, based on the insertion shape image generated by the insertion shape image generation unit 261, processing for obtaining a classification result that a kind of the insertion shape of the insertion portion 11 included in the insertion shape image is classified as one of a plurality of predetermined kinds. <Configuration of Insertion Shape Classification Unit 262>

A specific example of a configuration of the insertion shape classification unit 262 in the present embodiment will be described below.

In the present embodiment, the insertion shape classification unit 262 is configured to obtain a classification result that the kind of the insertion shape of the insertion portion 11 included in an insertion shape image generated by the insertion shape image generation unit 261 is classified as one of a plurality of predetermined kinds, by performing, for example, processing using a classifier (for example, classifier CLP) produced by learning each combination coefficient (weight) in a convolutional neural network (CNN) corresponding to a multi-layer neural network including an input layer, one or more convolutional layers, and an output layer by a learning method such as deep learning.

At production of the classifier CLP described above, for example, machine learning is performed by using teacher data including an insertion shape image and a label, the insertion shape image being similar to the insertion shape image generated by the insertion shape image generation unit 261, the label indicating a classification result that the insertion shape of the insertion portion 11 included in the insertion shape image is classified as one of a plurality of predetermined kinds.

The above-described plurality of predetermined kinds are each set, for example, as a kind of an insertion shape among various insertion shapes that can be formed in a duration from a time point at which insertion of the insertion portion 11 into the subject starts to a time point at which insertion of the insertion portion 11 into the subject ends, the insertion shape being a characteristic shape that affects determination of whether a manually or automatically performed insertion operation of the insertion portion 11 is successful and determination of whether it is needed to change an operation content.

At production of the above-described teacher data, for example, work is performed for applying, to one insertion shape image, a label in accordance with a determination result when a kind to which the insertion shape of the insertion portion 11 included in the one insertion shape image belongs among the plurality of predetermined kinds is visually determined by an experienced and skilled person.

Thus, with the above-described classifier CLP, for example, multi-dimensional data such as a pixel value of each pixel included in an insertion shape image generated by the insertion shape image generation unit 261 is acquired and inputted as input data to the input layer of the neural network, and accordingly, a plurality of likelihoods corresponding to respective kinds that would be classified as the kind of the insertion shape of the insertion portion 11 included in the insertion shape image can be acquired as output data to be outputted from the output layer of the neural network.

In addition, through the above-described processing using the classifier CLP, for example, one insertion shape kind corresponding to one highest likelihood among the plurality of likelihoods included in the output data outputted from the output layer of the neural network can be obtained as a classification result of the insertion shape of the insertion portion 11.

In other words, the insertion shape classification unit 262 is configured to obtain a classification result indicating the kind of the insertion shape of the insertion portion 11 inserted into the subject by performing processing using the classifier CLP produced by performing machine learning using teacher data including an insertion shape image and a label, the insertion shape image illustrating the insertion shape of the insertion portion 11, the label indicating a classification result that the insertion shape of the insertion portion 11 included in the insertion shape image is classified as one of a plurality of predetermined kinds.

A specific example of the classification result of the insertion shape of the insertion portion 11, which can be obtained through the above-described processing using the classifier CLP, will be described below. Note that the description will be made on an example in which a classification result is obtained in accordance with the kind of any insertion shape that appears between right before formation start of an α loop and right after of disentanglement completion among various insertion shapes that can be formed by the insertion portion 11 inserted into the subject.

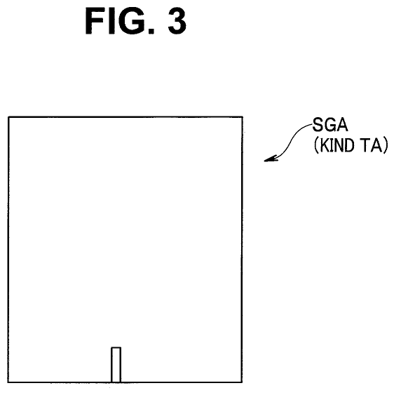

For example, the insertion shape classification unit 262 acquires a classification result that the insertion shape of the insertion portion 11 is classified as a kind TA by performing processing based on output data obtained by inputting, to the classifier CLP, a pixel value of each pixel included in an insertion shape image SGA as illustrated in FIG. 3. FIG. 3 is a diagram illustrating an example of an insertion shape image generated in the endoscope system according to the first embodiment.

The above-described kind TA is acquired as, for example, a classification result corresponding to a state in which the distal end portion 12 is positioned in an interval from a vicinity of the anus to a vicinity of the entrance of the sigmoid colon with the insertion portion 11 maintained in a substantially straight shape.

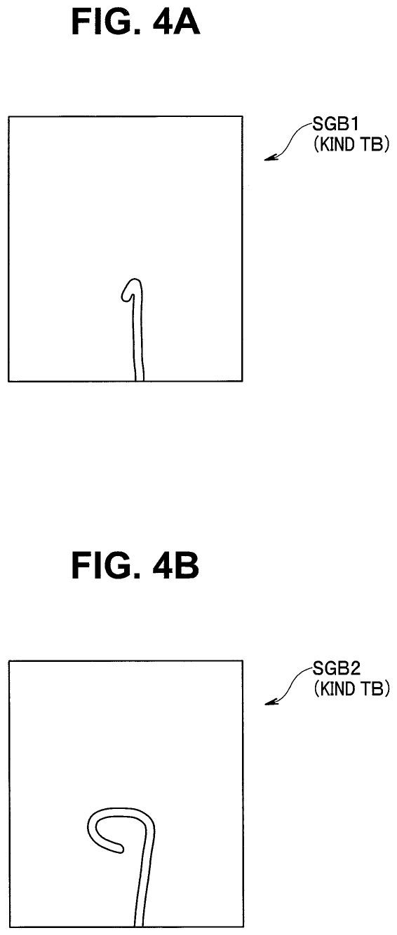

The insertion shape classification unit 262 acquires, for example, a classification result that the insertion shape of the insertion portion 11 is classified as a kind TB by performing processing based on output data obtained by inputting, to the classifier CLP, a pixel value of each pixel included in an insertion shape image SGB1 as illustrated in FIG. 4A or an insertion shape image SGB2 as illustrated in FIG. 4B. FIGS. 4A and 4B are each a diagram illustrating an example of an insertion shape image generated in the endoscope system according to the first embodiment.

The above-described kind TB is acquired as, for example, a classification result corresponding to a state in which the distal end portion 12 is positioned inside the sigmoid colon and the insertion portion 11 forms a curved shape that leads to an α loop.

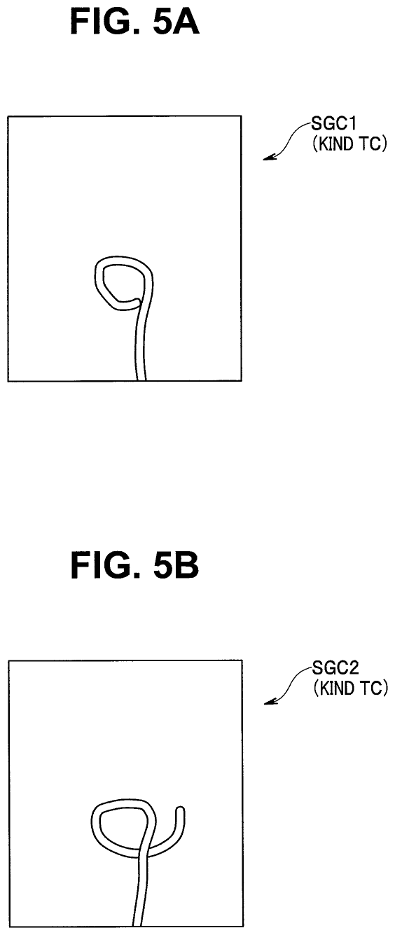

The insertion shape classification unit 262 acquires, for example, a classification result that the insertion shape of the insertion portion 11 is classified as a kind TC by performing processing based on output data obtained by inputting, to the classifier CLP, a pixel value of each pixel included in an insertion shape image SGC1 as illustrated in FIG. 5A or an insertion shape image SGC2 as illustrated in FIG. 5B. FIGS. 5A and 5B are each a diagram illustrating an example of an insertion shape image generated in the endoscope system according to the first embodiment.

The above-described kind TC is acquired as, for example, a classification result corresponding to a range of a state in which the distal end portion 12 has started forming an α loop by intersecting with any of the bending portion 13 or the flexible tube portion 14 to a state in which the distal end portion 12 has reached near an upper end part of the α loop.

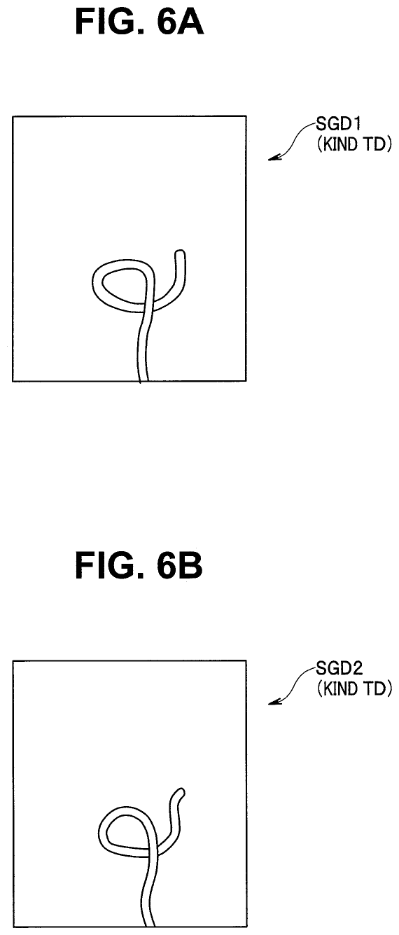

The insertion shape classification unit 262 acquires, for example, a classification result that the insertion shape of the insertion portion 11 is classified as a kind TD by performing processing based on output data obtained by inputting, to the classifier CLP, a pixel value of each pixel included in an insertion shape image SGD1 as illustrated in FIG. 6A or an insertion shape image SGD2 as illustrated in FIG. 6B. FIGS. 6A and 6B are each a diagram illustrating an example of an insertion shape image generated in the endoscope system according to the first embodiment.

The above-described kind TD is acquired as, for example, a classification result corresponding to a state in which the distal end portion 12 has reached a position slightly beyond the upper end part of the α loop.

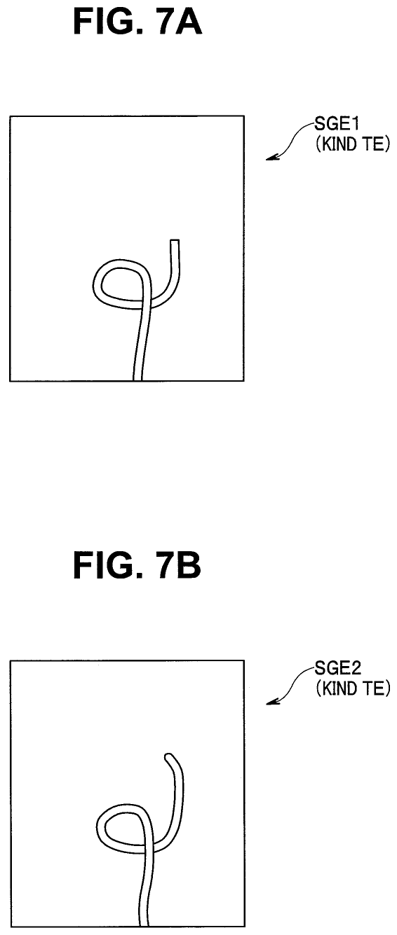

The insertion shape classification unit 262 acquires, for example, a classification result that the insertion shape of the insertion portion 11 is classified as a kind TE by performing processing based on output data obtained by inputting, to the classifier CLP, a pixel value of each pixel included in an insertion shape image SGE1 as illustrated in FIG. 7A or an insertion shape image SGE2 as illustrated in FIG. 7B. FIGS. 7A and 7B are each a diagram illustrating an example of an insertion shape image generated in the endoscope system according to the first embodiment.

The above-described kind TE is acquired as, for example, a classification result corresponding to any of a state in which the distal end portion 12 has reached near a splenic flexure and a state in which the distal end portion 12 has reached a position sufficiently separated from the upper end part of the α loop.

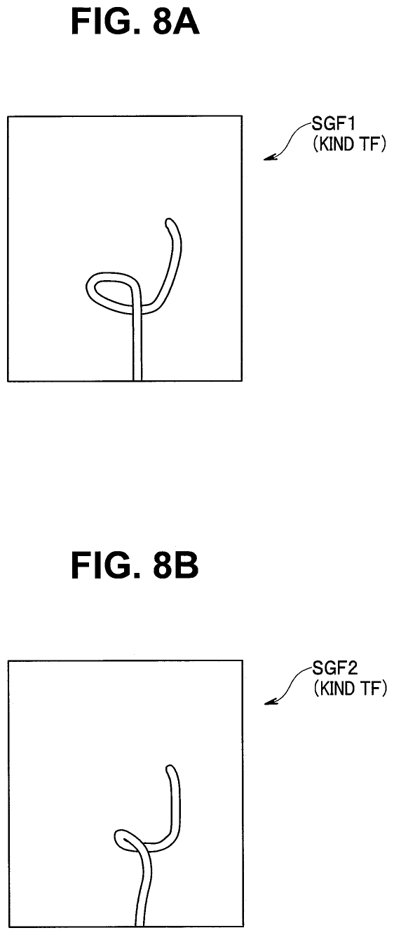

The insertion shape classification unit 262 acquires, for example, a classification result that the insertion shape of the insertion portion 11 is classified as a kind TF by performing processing based on output data obtained by inputting, to the classifier CLP, a pixel value of each pixel included in an insertion shape image SGF1 as illustrated in FIG. 8A or an insertion shape image SGF2 as illustrated in FIG. 8B. FIGS. 8A and 8B are each a diagram illustrating an example of an insertion shape image generated in the endoscope system according to the first embodiment.

The above-described kind TF is acquired as, for example, a classification result corresponding to a state in which the α loop has loosened along with progress of disentanglement of the α loop formed by the insertion portion 11.

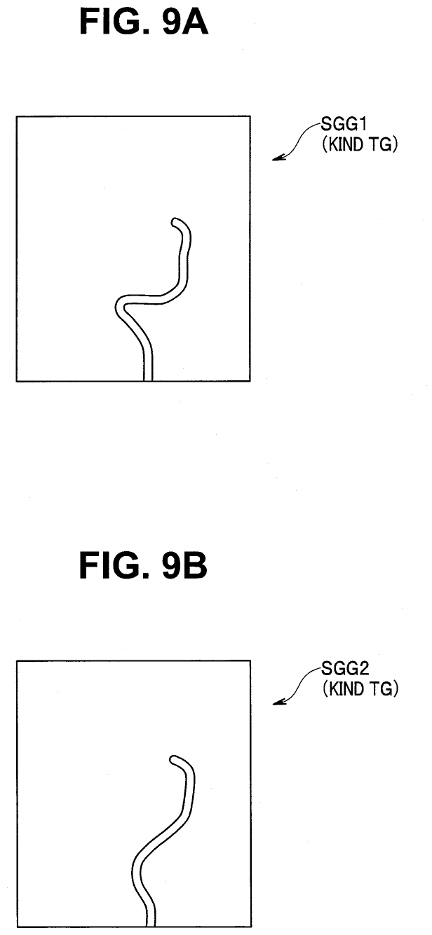

The insertion shape classification unit 262 acquires, for example, a classification result that the insertion shape of the insertion portion 11 is classified as a kind TG by performing processing based on output data obtained by inputting, to the classifier CLP, a pixel value of each pixel included in an insertion shape image SGG1 as illustrated in FIG. 9A or an insertion shape image SGG2 as illustrated in FIG. 9B. FIGS. 9A and 9B are each a diagram illustrating an example of an insertion shape image generated in the endoscope system according to the first embodiment.

The above-described kind TG is acquired as, for example, a classification result corresponding to a range of a state in which the α loop has transitioned to a shape similar to an N loop along with the progress of disentanglement of the α loop formed by the insertion portion 11 to a state right after the α loop is completely disentangled.

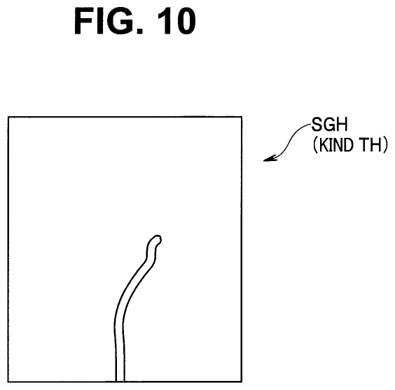

The insertion shape classification unit 262 acquires, for example, a classification result that the insertion shape of the insertion portion 11 is classified as a kind TH by performing processing based on output data obtained by inputting, to the classifier CLP, a pixel value of each pixel included in an insertion shape image SGH as illustrated in FIG. 10. FIG. 10 is a diagram illustrating an example of an insertion shape image generated in the endoscope system according to the first embodiment.

The above-described kind TH is acquired as, for example, a classification result corresponding to any of a state in which the distal end portion 12 has reached near the entrance of the transverse colon and a state in which the insertion portion 11 has transitioned to a substantially straight shape after disentanglement of the α loop.

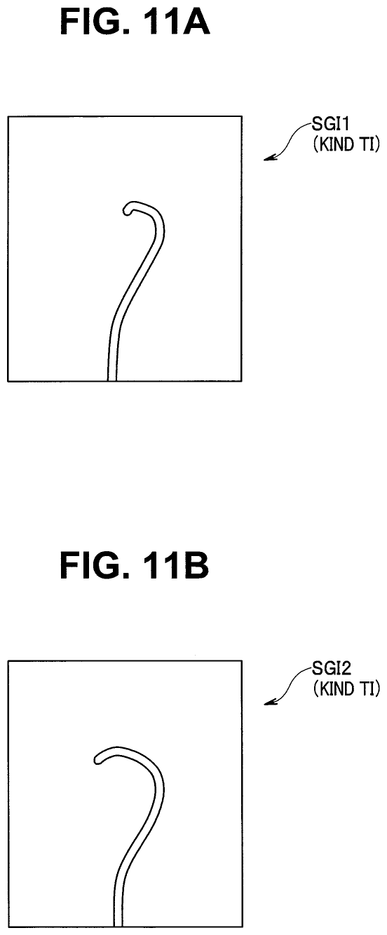

The insertion shape classification unit 262 acquires, for example, a classification result that the insertion shape of the insertion portion 11 is classified as a kind TI by performing processing based on output data obtained by inputting, to the classifier CLP, a pixel value of each pixel included in an insertion shape image SGI1 as illustrated in FIG. 11A or an insertion shape image SGI2 as illustrated in FIG. 11B. FIGS. 11A and 11B are each a diagram illustrating an example of an insertion shape image generated in the endoscope system according to the first embodiment.

The above-described kind TI is acquired as, for example, a classification result corresponding to a state in which the distal end portion 12 is positioned inside the transverse colon.

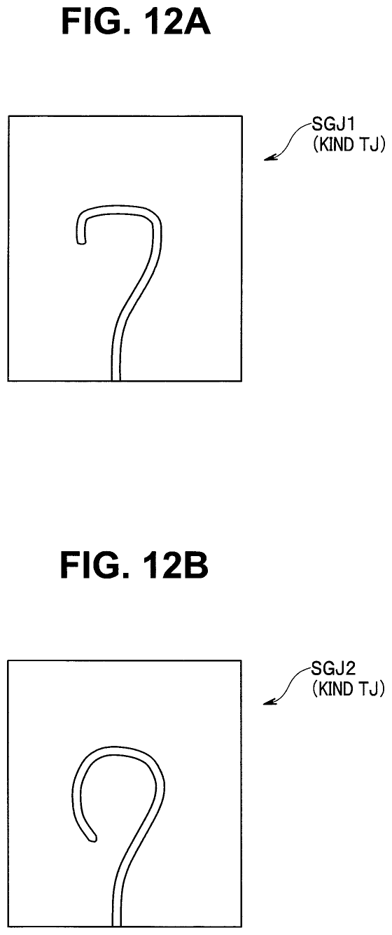

The insertion shape classification unit 262 acquires, for example, a classification result that the insertion shape of the insertion portion 11 is classified as a kind TJ by performing processing based on output data obtained by inputting, to the classifier CLP, a pixel value of each pixel included in an insertion shape image SGJ1 as illustrated in FIG. 12A or an insertion shape image SGJ2 as illustrated in FIG. 12B. FIGS. 12A and 12B are each a diagram illustrating an example of an insertion shape image generated in the endoscope system according to the first embodiment.

The above-described kind TJ is acquired as, for example, a classification result corresponding to a state in which the distal end portion 12 is positioned in an interval from the ascending colon to a vicinity of the cecum.

Note that, according to the present embodiment, for example, at production of the classifier CLP, a classification result in accordance with the kind of any insertion shape that appears between right before formation start of a shape different from an α loop and right after disentanglement completion may be obtained by performing learning by using an insertion shape image for which at least one kind among ten kinds of labels corresponding to the respective kinds TA to TJ is changed or by performing learning with an additional insertion shape image to which a label of a new kind different from any of the ten kinds of labels corresponding to the respective kinds TA to TJ is added.

Specifically, according to the present embodiment, for example, a classification result may be obtained in accordance with the kind of any insertion shape that appears between right before formation start of at least one shape among a reversed α loop, an inverted α loop, an N loop, a γ loop, and a stick shape and right after disentanglement completion.

Moreover, according to the present embodiment, for example, a classification result corresponding to the kind of any desired insertion shape that can be formed in a duration from a time point at which insertion of the insertion portion 11 into the subject is started to a time point at which insertion of the insertion portion 11 into the subject ends may be obtained by changing, as appropriate, a method of applying a label to a learning insertion shape image used at production of the classifier CLP.

The insertion control unit 263 is configured to generate an insertion control signal including information for performing control of an insertion operation of the insertion portion 11, based on at least one of an endoscope image outputted from the image processing unit 220, external force information outputted from the external force information acquisition device 40, or an insertion shape image generated by the insertion shape image generation unit 261, and based on a classification result obtained by the insertion shape classification unit 262, and output the insertion control signal to the endoscope function control unit 240.

Specifically, the insertion control unit 263 is configured to generate, based on at least one of an endoscope image outputted from the image processing unit 220, external force information outputted from the external force information acquisition device 40, or an insertion shape image generated by the insertion shape image generation unit 261 and based on a classification result obtained by the insertion shape classification unit 262, an insertion control signal including information for performing, as control of an insertion operation of the insertion portion 11, for example, control of at least one of start of the insertion operation, continuation of the insertion operation, interruption of the insertion operation, resumption of the insertion operation, stop of the insertion operation, or completion of the insertion operation, and is configured to output the insertion control signal to the endoscope function control unit 240.

The insertion control unit 263 is also configured to generate, based on at least one of an endoscope image outputted from the image processing unit 220, external force information outputted from the external force information acquisition device 40, or an insertion shape image generated by the insertion shape image generation unit 261 and based on a classification result obtained by the insertion shape classification unit 262, an insertion control signal including information for controlling at least one of an operation amount of an insertion operation of the insertion portion 11, operation speed of the insertion operation, or operation force of the insertion operation, and is configured to output the insertion control signal to the endoscope function control unit 240.

The insertion control unit 263 of the present embodiment is configured to be able to, for example, set a control content in accordance with a kind of the current insertion shape of the insertion portion 11, which is indicated as a classification result obtained by the insertion shape classification unit 262, based on at least one of an endoscope image outputted from the image processing unit 220, external force information outputted from the external force information acquisition device 40, or an insertion shape image generated by the insertion shape image generation unit 261, generate an insertion control signal including information for performing control of an insertion operation of the insertion portion 11 by using the set control content, and output the insertion control signal to the endoscope function control unit 240.

Thus, the insertion control unit 263 can set, for example, an operation control group CGA including a control content for performing an insertion operation of the insertion portion 11 by executing alone a basic operation selected from among basic operations achieved by respective functions of the endoscope 10, in accordance with the kind of the current insertion shape of the insertion portion 11, which is indicated as a classification result obtained by the insertion shape classification unit 262, based on at least one of an endoscope image outputted from the image processing unit 220, external force information outputted from the external force information acquisition device 40, or an insertion shape image generated by the insertion shape image generation unit 261, and generate and output an insertion control signal including information of the set operation control group CGA.

Specifically, the operation control group CGA includes, for example, control contents of a forward movement amount, forward movement speed, operation force, and the like when a pushing operation is executed.

Moreover, the insertion control unit 263 can set an operation control group CGB including a control content for performing an insertion operation of the insertion portion 11 by executing, for example, a combination of a plurality of basic operations selected from among the basic operations achieved by respective functions of the endoscope 10, in accordance with the kind of the current insertion shape of the insertion portion 11, which is indicated as a classification result obtained by the insertion shape classification unit 262, based on at least one of an endoscope image outputted from the image processing unit 220, external force information outputted from the external force information acquisition device 40, or an insertion shape image generated by the insertion shape image generation unit 261, and generate and output an insertion control signal including information of the set operation control group CGB.

Specifically, the operation control group CGB includes, for example, control contents of a backward movement amount, backward movement speed, a rotational angle, a rotational direction, operation force, and the like when a combination of a pulling operation and a twisting operation is executed.

Note that the operation control group CGB is set as a control content for consecutively or simultaneously executing a plurality of basic operations selected from among the basic operations achieved by respective functions of the endoscope 10. In other words, the control content of the operation control group CGB is set as a more complicate control content than the control content of the operation control group CGA.

In other words, the insertion control unit 263 is configured to perform, as control in accordance with the kind of the current insertion shape of the insertion portion 11, which is indicated as a classification result obtained by the insertion shape classification unit 262, control based on one of the operation control group CGA including a control content for performing an insertion operation of the insertion portion 11 by executing alone a basic operation selected from among the basic operations achieved by respective functions of the endoscope 10 and the operation control group CGB including a control content for performing an insertion operation of the insertion portion 11 by executing a combination of a plurality of basic operations selected from among the basic operations achieved by respective functions of the endoscope 10.

In addition, the insertion control unit 263 performs control of an insertion operation of the insertion portion 11 based on at least one of an image obtained through image pickup of inside of the subject by the endoscope 10, information indicating magnitude of external force applied to the insertion portion 11, or information indicating the insertion shape of the insertion portion 11, and based on a classification result obtained by the insertion shape classification unit 262.

The classification result recording unit 264 is configured to be able to perform operation for recording, in time series, classification results obtained by the insertion shape classification unit 262.

In the present embodiment, at least some of functions of the main body device 20 may be achieved by the processor 20P. In addition, in the present embodiment, at least part of the main body device 20 may be configured as an individual electronic circuit or may be configured as a circuit block in an integrated circuit such as a field programmable gate array (FPGA).

In addition, a configuration according to the present embodiment may be modified as appropriate so that, for example, a computer reads a program for executing at least some of functions of the main body device 20 from the storage medium 20M such as a memory and performs operation in accordance with the read program.

As illustrated in FIG. 2, the insertion shape detection device 30 includes a reception antenna 310 and an insertion shape information acquisition unit 320.

The reception antenna 310 includes, for example, a plurality of coils for three-dimensionally detecting a magnetic field generated from each of the plurality of source coils 18. The reception antenna 310 is configured to detect a magnetic field generated from each of the plurality of source coils 18, generate a magnetic field detection signal in accordance with intensity of the detected magnetic field, and output the magnetic field detection signal to the insertion shape information acquisition unit 320.

The insertion shape information acquisition unit 320 is configured to acquire the position of each of the plurality of source coils 18 based on the magnetic field detection signal outputted from the reception antenna 310. The insertion shape information acquisition unit 320 is also configured to generate insertion shape information indicating the position of each of the plurality of source coils 18, which is acquired as described above, and output the insertion shape information to the insertion shape image generation unit 261.

Specifically, the insertion shape information acquisition unit 320 acquires, as the positions of the plurality of source coils 18, for example, a plurality of three-dimensional coordinate values in a spatial coordinate system virtually set with an origin or a reference point at a predetermined position (such as the anus) in the subject into which the insertion portion 11 is inserted. In addition, the insertion shape information acquisition unit 320 generates insertion shape information including the plurality of three-dimensional coordinate values acquired as described above and outputs the insertion shape information to the insertion shape image generation unit 261.

Then, in such a case, the insertion shape image generation unit 261 performs, for example, processing for acquiring a plurality of two-dimensional coordinate values corresponding to the plurality of respective three-dimensional coordinate values included in the insertion shape information outputted from the insertion shape information acquisition unit 320, processing for interpolating the acquired plurality of two-dimensional coordinate values, and processing for generating an insertion shape image in accordance with the plurality of interpolated two-dimensional coordinate values.

In the present embodiment, at least part of the insertion shape detection device 30 may be configured as an electronic circuit or may be configured as a circuit block in an integrated circuit such as a field programmable gate array (FPGA). In addition, in the present embodiment, for example, the insertion shape detection device 30 may include at least one processor (such as CPU).

According to the present embodiment, for example, when the insertion shape image generation unit 261 is configured to generate a three-dimensional insertion shape image three-dimensionally illustrating the insertion shape of the insertion portion 11 inserted into the subject, the classifier CLP of the insertion shape classification unit 262 may be configured to classify the kind of the insertion shape of the insertion portion 11 by using, as input data, multi-dimensional data such as pixel values acquired from the three-dimensional insertion shape image. In such a case, the classifier CLP may be produced by using, for example, a 3D convolutional neural network (3D-CNN).

According to the present embodiment, for example, the classifier CLP of the insertion shape classification unit 262 may be configured to classify the kind of the insertion shape of the insertion portion 11 by using, as input data, the plurality of three-dimensional coordinate values included in the insertion shape information outputted from the insertion shape detection device 30. In such a case, the classifier CLP may be produced by using a method of classifying the kind of the insertion shape of the insertion portion 11 by using numerical values as feature values, such as a well-known linear discriminant function or a well-known neural network.

According to the present embodiment, for example, at production of the classifier CLP, a label indicating a classification result that the insertion shape of the insertion portion 11 is classified as one of a plurality of predetermined kinds may be applied to an insertion shape image, and machine learning may be performed by using teacher data including the label and a plurality of three-dimensional coordinate values used at generation of the insertion shape image.

Subsequently, effects of the present embodiment will be described below. Note that the description will be made on an example in which control of an insertion operation of the insertion portion 11 inserted into the intestinal canal of the large intestine through the anus is performed. The description will be made also on an example in which an α loop is formed by the insertion portion 11 inserted into the intestinal canal.

A user such as a surgeon connects components of the endoscope system 1 and powers on the endoscope system 1, and then disposes the insertion portion 11 so that, for example, the distal end portion 12 is positioned near the anus or rectum of a subject.

According to an operation by the user as described above, an object is irradiated with illumination light supplied from the light source unit 210, image pickup of the object irradiated with the illumination light is performed by the image pickup unit 110, and an endoscope image obtained through the image pickup of the object is outputted from the image processing unit 220 to the display control unit 250 and the system control unit 260.

In addition, according to an operation by the user as described above, a coil drive signal is supplied from the coil drive signal generation unit 230, a magnetic field is generated by each of the plurality of source coils 18 in accordance with the coil drive signal, insertion shape information obtained by detecting the magnetic field is outputted from the insertion shape information acquisition unit 320 to the system control unit 260, and an insertion shape image in accordance with the insertion shape information is generated by the insertion shape image generation unit 261.

In addition, according to an operation by the user as described above, external force information indicating the magnitude and direction of external force at the position of each of the plurality of source coils 18 is outputted from the external force information acquisition device 40 to the system control unit 260.

In a state in which the insertion portion 11 is disposed as described above, for example, the user turns on an automatic insertion switch (not illustrated) of the input device 50 to provide an instruction for starting insertion control of the insertion portion 11 by the main body device 20.

When having detected the instruction for starting insertion control of the insertion portion 11, the classification result recording unit 264 starts, for example, operation for recording, in time series and at each constant time, classification results obtained by the insertion shape classification unit 262.

The insertion control unit 263 sets a control content in accordance with the kind of the current insertion shape of the insertion portion 11, which is indicated as a classification result obtained by the insertion shape classification unit 262, based on at least one of an endoscope image outputted from the image processing unit 220, external force information outputted from the external force information acquisition device 40, or an insertion shape image generated by the insertion shape image generation unit 261.

Specifically, for example, when having detected that the kind of the current insertion shape of the insertion portion 11, which is indicated as a classification result obtained by the insertion shape classification unit 262, is any of the kinds TA, TH, TI, and TJ, the insertion control unit 263 generates and outputs an insertion control signal including information of the operation control group CGA including a control content set based on at least one of an endoscope image outputted from the image processing unit 220, external force information outputted from the external force information acquisition device 40, or an insertion shape image generated by the insertion shape image generation unit 261.

For example, when having detected that the kind of the current insertion shape of the insertion portion 11, which is indicated as a classification result obtained by the insertion shape classification unit 262, is any of the kinds TB, TC, TD, TE, TF, and TG, the insertion control unit 263 generates and outputs an insertion control signal including information of the operation control group CGB including a control content set based on at least one of an endoscope image outputted from the image processing unit 220, external force information outputted from the external force information acquisition device 40, or an insertion shape image generated by the insertion shape image generation unit 261.

In other words, according to a specific example described above, when having detected that the kind of the current insertion shape of the insertion portion 11 does not correspond to the kind of any insertion shape that appears between right before formation start of an α loop and right after disentanglement completion, the insertion control unit 263 generates and outputs an insertion control signal including information of the operation control group CGA.

In addition, according to the specific example described above, when having detected that the kind of the current insertion shape of the insertion portion 11 corresponds to the kind of any insertion shape that appears between right before formation start of an α loop and right after disentanglement completion, the insertion control unit 263 generates and outputs an insertion control signal including information of the operation control group CGB including a more complicate control content than the control content of the operation control group CGA.

Note that, in the present embodiment, for example, the insertion control unit 263 may perform processing using a classifier CLQ to be described later when setting a control content based on an endoscope image outputted from the image processing unit 220.

Moreover, in the present embodiment, based on a processing result image PRG to be described later, which is obtained through the processing using the classifier CLQ, the insertion control unit 263 may set a control content for moving forward the insertion portion 11 by a relatively large forward movement amount, for example, when a lumen region exists at a central portion of the processing result image PRG, and may set a control content for moving forward the insertion portion 11 by a relatively small forward movement amount, for example, when a lumen region exists at a peripheral portion of the processing result image PRG.

For example, after having checked that the insertion shape of the insertion portion 11 inserted inside the subject has stopped changing based on an insertion shape image displayed on the display device 60, the user turns off the automatic insertion switch of the input device 50 to provide an instruction for stopping insertion control of the insertion portion 11 by the main body device 20.

When having detected the instruction for stopping insertion control of the insertion portion 11, the classification result recording unit 264 stops operation for recording, in time series and at each constant time, classification results obtained by the insertion shape classification unit 262.

When an examination is performed by inserting the insertion portion of the endoscope into the intestinal canal of the large intestine, various situations can occur in accordance with a combination of a state of progress in the large intestine, the insertion shape of the insertion portion, an insertion length of the insertion portion, and the like. When manually performing an insertion operation of the insertion portion of the endoscope, an experienced and skilled doctor determines magnitude of force applied to the insertion portion, a kind of an operation performed on the insertion portion, and the like as appropriate in accordance with a determination result of situation determination on a current situation.

With a conventional proposal related to automation of an insertion operation of the insertion portion of the endoscope, it is extremely difficult to acquire a determination result equivalent to a determination result of subjective situation determination by an experienced and skilled doctor as described above and perform control in accordance with the acquired determination result, which has been a problem.

However, according to the present embodiment, the insertion shape classification unit 262 performs processing to obtain a classification result by classifying the kind of the insertion shape of the insertion portion 11 included in an insertion shape image generated by the insertion shape image generation unit 261, based on a viewpoint substantially equivalent to a viewpoint when an experienced and skilled person subjectively determines or evaluates whether an operation is successful and the like in an insertion operation of the insertion portion 11.

Moreover, according to the present embodiment, the insertion control unit 263 performs insertion control in accordance with the kind of the insertion shape of the insertion portion 11, which is indicated as a classification result obtained by the insertion shape classification unit 262. Thus, according to the present embodiment, it is possible to perform appropriate insertion control in accordance with an insertion situation of the insertion portion, such as individual difference in an internal state of a subject into which the insertion portion is inserted or temporal change of the insertion shape of the insertion portion inside the subject.

According to conventional endoscope observation using a device having functions same as functions of the insertion shape detection device 30, information related to the insertion shape of the insertion portion of the endoscope can be recorded during observation in a subject, but reuse of the information after the observation in the subject ends is not assumed, which is a problem. Thus, according to conventional endoscope observation using a device having functions same as functions of the insertion shape detection device 30, it is difficult to, for example, evaluate or analyze transition of the insertion shape of the insertion portion of the endoscope during observation in a subject, after the observation in the subject ends, which is another problem attributable to the above-described problem.

However, according to processing and the like of the present embodiment as described above, classification results obtained by the insertion shape classification unit 262 are recorded in time series in the classification result recording unit 264 in a duration until the automatic insertion switch of the input device 50 is turned off after turned on. Thus, according to the present embodiment, it is possible to, for example, evaluate or analyze transition of the insertion shape of the insertion portion 11 during observation in a subject, after the observation in the subject ends, by using information recorded in the classification result recording unit 264.

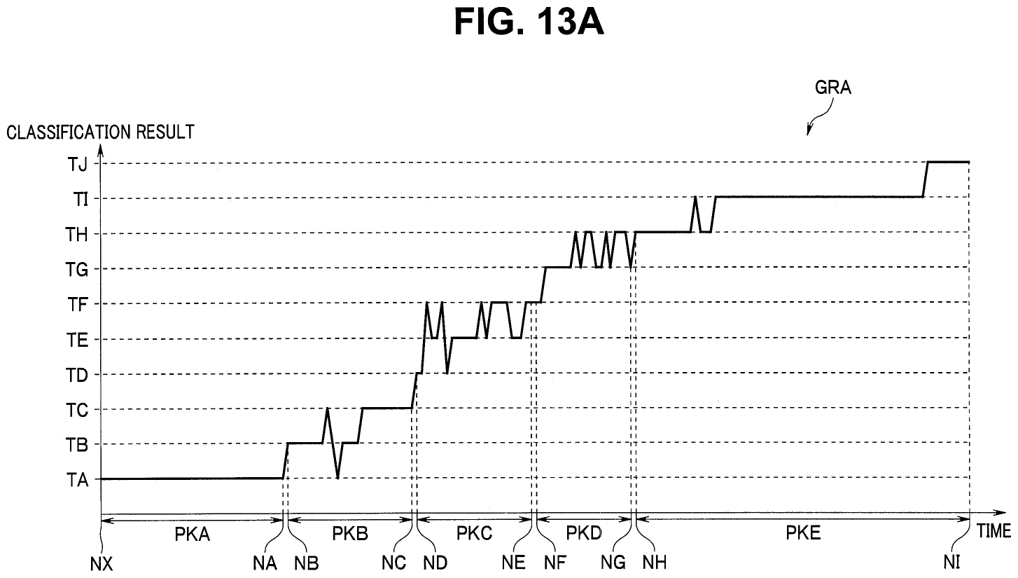

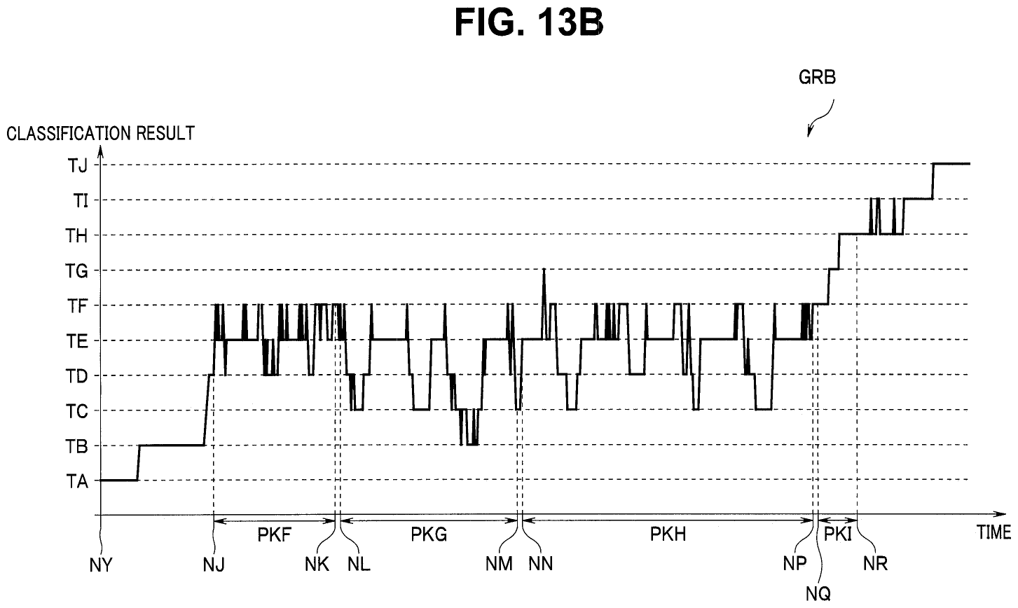

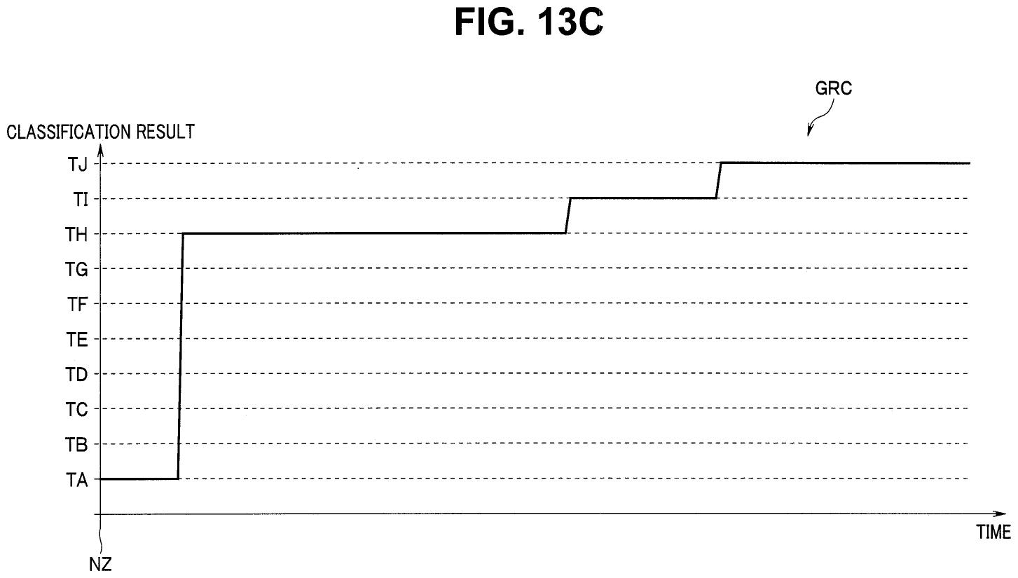

Specifically, for example, the display control unit 250 performs processing for visualizing information recorded in the classification result recording unit 264, thereby displaying, on the display device 60, a display image including a graph indicating temporal transition of the kind of the insertion shape of the insertion portion 11, which is obtained as a result of classification by the insertion shape classification unit 262, as illustrated in FIGS. 13A to 13C. FIGS. 13A to 13C are each a diagram illustrating an example in which temporal transition of the kind of the insertion shape of the insertion portion is visualized by using information recorded in the endoscope system according to the first embodiment.

A graph GRA in FIG. 13A is produced as a graph indicating temporal transition of the kind of the insertion shape of the insertion portion 11 when the distal end portion 12 is moved to the ascending colon along with disentanglement of the a loop formed by the insertion portion 11.

According to the graph GRA in FIG. 13A, the kind of the insertion shape of the insertion portion 11 is maintained as the kind TA in a duration PKA corresponding to a duration from a time point NX at which insertion control of the insertion portion 11 is started to a time point NA. Thus, according to the graph GRA in FIG. 13A, for example, it can be checked that the distal end portion 12 reaches near the entrance of the sigmoid colon in the duration PKA in a state in which the insertion portion 11 is maintained in a substantially straight shape.

According to the graph GRA in FIG. 13A, the kind of the insertion shape of the insertion portion 11 changes from the kind TB to the kind TC in a duration PKB corresponding to a duration from a time point NB to a time point NC after the time point NA. Thus, according to the graph GRA in FIG. 13A, for example, it can be checked that the insertion portion 11 starts forming an α loop in the duration PKB.

According to the graph GRA in FIG. 13A, the kind of the insertion shape of the insertion portion 11 changes from the kind TD to the kind TG in a duration PKC corresponding to a duration from a time point ND to a time point NE after the time point NC. In addition, according to the graph GRA in FIG. 13A, the kind of the insertion shape of the insertion portion 11 oscillatorily changes to any of the kinds TE and TF halfway through the duration PKC. Thus, according to the graph GRA in FIG. 13A, for example, it can be checked that the α loop is being disentangled with an attempt to loosen the α loop formed by the insertion portion 11 in the duration PKC.

According to the graph GRA in FIG. 13A, the kind of the insertion shape of the insertion portion 11 changes from the kind TF to the kind TG and then oscillatorily changes to any of the kinds TG and TH in a duration PKD corresponding to a duration from a time point NF to a time point NG after the time point NE. Thus, according to the graph GRA in FIG. 13A, for example, it can be checked that the disentanglement of the α loop formed by the insertion portion 11 is about to be completed in the duration PKD.

According to the graph GRA in FIG. 13A, the kind of the insertion shape of the insertion portion 11 changes from the kind TH to the kind TJ through the kind TI in a duration PKE corresponding to a duration from a time point NH to a time point NI after the time point NG. Thus, according to the graph GRA in FIG. 13A, for example, it can be checked that the distal end portion 12 reaches the ascending colon through the transverse colon in the duration PKE.

A graph GRB in FIG. 13B is produced as a graph indicating temporal transition of the kind of the insertion shape of the insertion portion 11 when it is difficult to disentangle an α loop due to, for example, individual difference in a shape of the sigmoid colon. Note that, for convenience of illustration, scaling of a horizontal axis in FIG. 13B is different from scaling of a horizontal axis in FIGS. 13A and 13C.

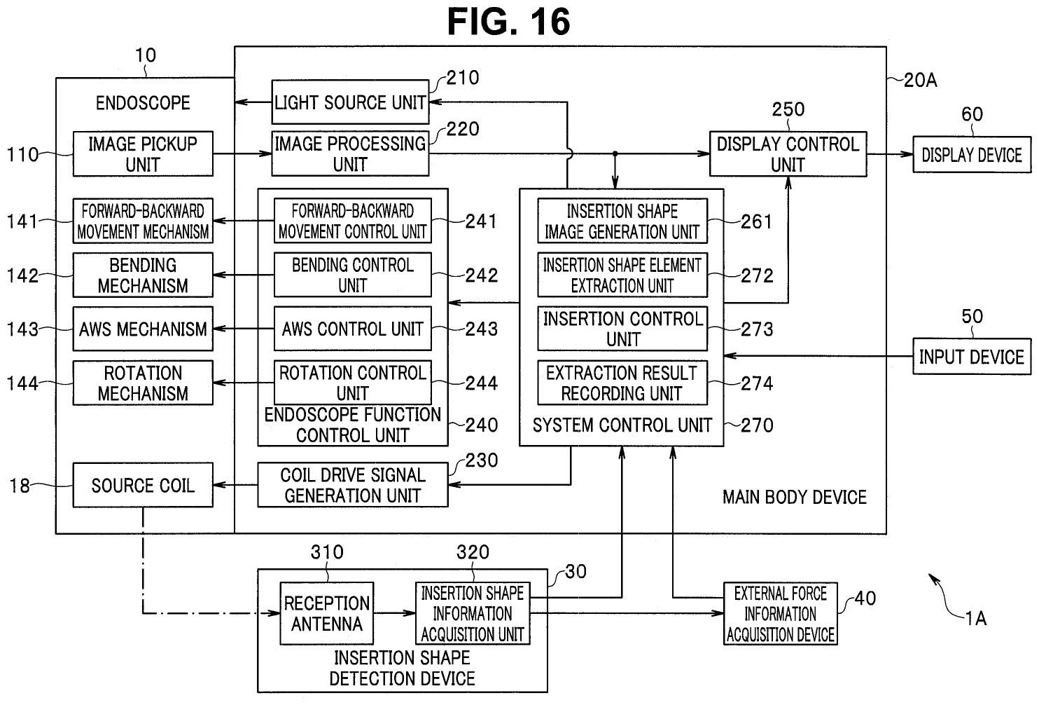

According to the graph GRB in FIG. 13B, the kind of the insertion shape of the insertion portion 11 oscillatorily changes to any of the kinds TD, TE, and TF in a duration PKF corresponding to a duration from a time point NJ to a time point NK after a time point NY at which insertion control of the insertion portion 11 is started. Thus, according to the graph GRB in FIG. 16B, for example, it can be checked that an attempt to loosen the α loop formed by the insertion portion 11 is not successful in the duration PKF.

According to the graph GRB in FIG. 13B, the kind of the insertion shape of the insertion portion 11 oscillatorily changes to any of the kinds TB, TC, TD, TE, and TF in a duration PKG corresponding to a duration from a time point NL to a time point NM after the time point NK. Thus, according to the graph GRB in FIG. 13B, for example, it can be checked that reformation of an α loop by the insertion portion 11 is performed in the duration PKG in a state in which a shape of the intestinal canal is prepared not to interfere with insertion of the insertion portion 11 as much as possible.

According to the graph GRB in FIG. 13B, the kind of the insertion shape of the insertion portion 11 oscillatorily changes to any of the kinds TC, TD, TE, TF, and TG in a duration PKH corresponding to a duration from a time point NN to a time point NP after the time point NM. Thus, according to the graph GRB in FIG. 13B, for example, it can be checked that disentanglement of the α loop reformed by the insertion portion 11 is attempted in the duration PKH.

According to the graph GRB in FIG. 13B, the kind of the insertion shape of the insertion portion 11 changes from the kind TF to the kind TH through the kind TG in a duration PKI corresponding to a duration from a time point NQ to a time point NR after the time point NP. Thus, according to the graph GRB in FIG. 13B, for example, it can be checked that disentanglement of the α loop reformed by the insertion portion 11 is successful in the duration PKI.

A graph GRC in FIG. 13C is produced as a graph indicating temporal transition of the kind of the insertion shape of the insertion portion 11 when the distal end portion 12 is moved to the ascending colon in a state in which no α loop is formed by the insertion portion 11.

According to the graph GRC in FIG. 13C, the kind of the insertion shape of the insertion portion 11 changes in an order of the kinds TA, TH, TI, and TJ after a time point NZ at which insertion control of the insertion portion 11 is started. Thus, according to the graph GRC in FIG. 13C, for example, it can be checked that no problem that interferes with insertion of the insertion portion 11 occurs in an entire interval of the large intestine.

According to the present embodiment, the classification result recording unit 264 may perform operation for recording, in time series and at each constant time, classification results obtained by the insertion shape classification unit 262, not only in a case in which the insertion portion 11 is automatically inserted by control of the insertion control unit 263 but also in a case in which the insertion portion 11 is manually inserted through an operation by the user. When such operation by the classification result recording unit 264 is performed at manual insertion of the insertion portion 11, graphs similar to the graphs exemplarily illustrated in FIGS. 13A to 13C can be produced as graphs indicating temporal transition of the kind of the insertion shape of the insertion portion 11 due to an operation by the user.

In addition, when the operation of the classification result recording unit 264 as described above is performed at manual insertion of the insertion portion 11, for example, it is possible to acquire data that is usable when an insertion operation of the insertion portion 11, which is performed by the user, is quantitatively evaluated and/or analyzed.

According to the present embodiment, the classification result recording unit 264 may perform operation for recording, in time series and at each constant time, classification results obtained by the insertion shape classification unit 262, not only in a case in which the insertion portion 11 is inserted into the subject, but also in a case in which the insertion portion 11 inserted into the subject is removed.

According to the present embodiment, information recorded in the classification result recording unit 264 may be used for usage other than production of graphs as exemplarily illustrated in FIGS. 13A to 13C.

Specifically, information recorded in the classification result recording unit 264 can be used as, for example, original data in analytical methods such as data mining and statistical analysis. Information recorded in the classification result recording unit 264 can be also used for, for example, evaluation of skills of the user when the insertion portion 11 is manually inserted through an operation by the user. In addition, information recorded in the classification result recording unit 264 can be used for, for example, estimation of insertion difficulty when the insertion portion 11 is inserted into a certain subject.

According to the present embodiment, the classification result recording unit 264 may be configured to perform, for example, operation for recording desired information, such as an endoscope image, which can be obtained through operation of the endoscope system 1, in association with a classification result obtained by the insertion shape classification unit 262.

Note that, for example, the insertion control unit 263 of the present embodiment may be configured to set a control content in accordance with a detection result obtained by detecting whether the kind of the insertion shape of the insertion portion 11, which is indicated as a classification result obtained by the insertion shape classification unit 262, has changed, based on at least one of an endoscope image outputted from the image processing unit 220, external force information outputted from the external force information acquisition device 40, or an insertion shape image generated by the insertion shape image generation unit 261, generate an insertion control signal including information for performing control of an insertion operation of the insertion portion 11 by using the set control content, and output the insertion control signal to the endoscope function control unit 240.

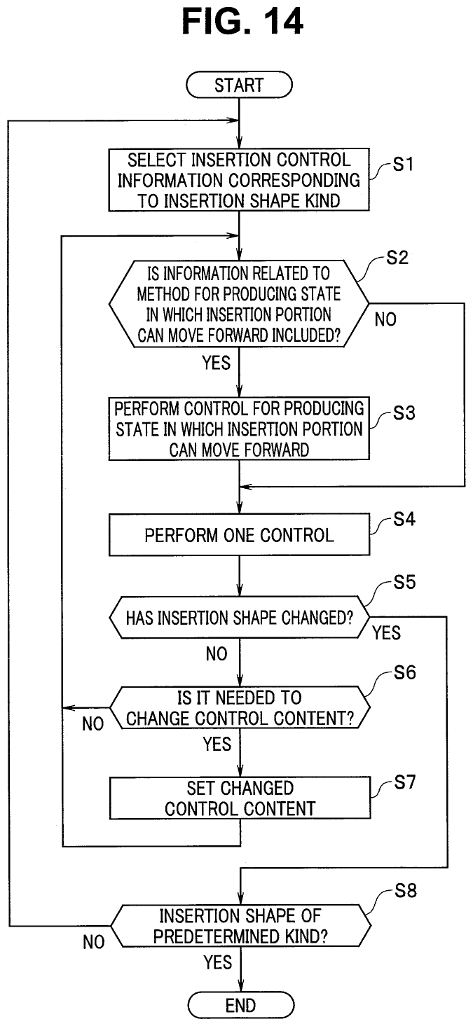

Specifically, the insertion control unit 263 may be configured to perform, for example, control as illustrated in FIG. 14. An outline of such control will be described below. Note that, for simplification, the description below will be made on an example in which a plurality of pieces of insertion control information produced as information including control contents corresponding to a plurality of predetermined kinds of insertion shapes classified by the insertion shape classification unit 262 are stored in the storage medium 20M in advance, one piece of insertion control information in accordance with a classification result obtained by the insertion shape classification unit 262 is selected from among the plurality of pieces of insertion control information, and whether the kind of the insertion shape of the insertion portion 11, which is indicated as the classification result, has changed is detected at each execution of control by the insertion control unit 263. FIG. 14 is a flowchart for description of an outline of control performed in an endoscope system according to a modification of the first embodiment.

The insertion control unit 263 performs, based on a classification result obtained by the insertion shape classification unit 262, processing for selecting and reading one piece of insertion control information corresponding to one insertion shape kind indicated as the classification result from among a plurality of pieces of insertion control information stored in the storage medium 20M in advance (step S1 in FIG. 14).

Each of the above-described plurality of pieces of insertion control information includes either information related to a method for producing a state in which the insertion portion 11 can move forward or information related to a method for disentangling a certain insertion shape formed by the insertion portion 11. Each of the above-described plurality of pieces of insertion control information also includes information indicating control content for one time (such as control amount) corresponding to operation of at least one control unit among the control units included in the endoscope function control unit 240, in other words, at least one basic operation among the basic operations achieved by respective functions of the endoscope 10.

The information related to a method for producing a state in which the insertion portion 11 can move forward includes information indicating a setting condition for setting a movement destination of the distal end portion 12, for example, a frame WG set to the processing result image PRG, which will be described later. The information related to a method for producing a state in which the insertion portion 11 can move forward also includes, for example, at least one of information indicating a basic operation that is executed alone at forward movement of the insertion portion 11 among the basic operations achieved by respective functions of the endoscope 10 or information indicating a combination of a plurality of basic operations that are consecutively or simultaneously executed at forward movement of the insertion portion 11 among the basic operations.

The information related to a method for disentangling a certain insertion shape formed by the insertion portion 11 includes, for example, at least one of information indicating a basic operation that is individually executed at disentangle of the certain insertion shape among the basic operations achieved by respective functions of the endoscope 10 or information indicating a combination of a plurality of basic operations that are consecutively or simultaneously executed at disentangle of the certain insertion shape among the basic operations.

The insertion control unit 263 detects whether the information related to a method for producing a state in which the insertion portion 11 can move forward is included in the one piece of insertion control information read at step Si in FIG. 14 (step S2 in FIG. 14).

When having acquired a detection result that the information related to a method for producing a state in which the insertion portion 11 can move forward is not included in the one piece of insertion control information read at step S1 in FIG. 14 (NO at S2), the insertion control unit 263 performs processing at step S4 in FIG. 14 to be described later.

When having acquired a detection result that the information related to a method for producing a state in which the insertion portion 11 can move forward is included in the one piece of insertion control information read at step S1 in FIG. 14 (YES at S2), the insertion control unit 263 performs control for producing a state in which the insertion portion 11 can move forward on the endoscope function control unit 240 based on a control content included in the one piece of insertion control information and an endoscope image outputted from the image processing unit 220 (step S3 in FIG. 14).

The insertion control unit 263 generates an insertion control signal for performing one control in accordance with any of the control content included in the one piece of insertion control information read at step S1 in FIG. 14 or a changed control content that is set at step S6 in FIG. 14 to be described later, based on, for example, external force information outputted from the external force information acquisition device 40, and outputs the insertion control signal to the endoscope function control unit 240 (step S4 in FIG. 14). Note that a specific example of the above-described one control will be described later.

The insertion control unit 263 compares a classification result obtained by the insertion shape classification unit 262 at a timing when the processing at step S1 in FIG. 14 is performed and a classification result obtained by the insertion shape classification unit 262 at a timing right after one control is performed at step S4 in FIG. 14, thereby detecting whether the kind of the insertion shape of the insertion portion 11 has changed in accordance with the one control (step S5 in FIG. 14).

When having acquired a detection result that the kind of the insertion shape of the insertion portion 11 has changed (YES at S5), the insertion control unit 263 performs processing at step S8 in FIG. 14 to be described later. When having acquired a detection result that the kind of the insertion shape of the insertion portion 11 has not changed (NO at S5), the insertion control unit 263 determines whether it is needed to change a control content when one control is performed at step S4 in FIG. 14 (step S6 in FIG. 14).

When having acquired a determination result that it is not needed to change the control content when one control is performed at step S4 in FIG. 14 (NO at S6), the insertion control unit 263 maintains the control content and performs the above-described control at step S2 in FIG. 14 or later. When having acquired a determination result that it is needed to change the control content when one control is performed at step S4 in FIG. 14 (YES at S6), the insertion control unit 263 performs processing for setting a changed control content (step S7 in FIG. 14) and then performs the above-described control at step S2 in FIG. 14 or later.

The insertion control unit 263 detects whether the insertion shape of the insertion portion 11 has changed to a predetermined kind based on a classification result obtained by the insertion shape classification unit 262 at a timing right after one control is performed at step S4 in FIG. 14 (step S8 in FIG. 14).

When having acquired a detection result that the insertion shape of the insertion portion 11 has not changed to the predetermined kind (NO at S8), the insertion control unit 263 performs the above-described control at step S1 in FIG. 14. When having acquired a detection result that the insertion shape of the insertion portion 11 has changed to the predetermined kind (YES at S8), the insertion control unit 263 ends the series of controls on the endoscope function control unit 240.

In other words, according to the series of pieces of processing in FIG. 14, the insertion control unit 263 is configured to set a control content in accordance with one insertion shape kind indicated as a classification result obtained by the insertion shape classification unit 262, perform one insertion control of an insertion operation of the insertion portion 11 based on the set control content, and determine, each time the one insertion control is performed, whether to change a control content of the insertion control by referring to a classification result obtained by the insertion shape classification unit 262.

In addition, according to the series of pieces of processing in FIG. 14, the insertion control unit 263 is configured to select insertion control information CJX including a control content corresponding to an insertion shape of a kind TX indicated as a classification result obtained by the insertion shape classification unit 262 from among a plurality of pieces of insertion control information stored in the storage medium 20M in advance.

Moreover, according to the series of pieces of processing in FIG. 14, the insertion control unit 263 is configured to perform one insertion control based on the control content included in the insertion control information CJX. In addition, according to the series of pieces of processing in FIG. 14, when having detected that the kind of the insertion shape of the insertion portion 11, which is indicated as a classification result obtained by the insertion shape classification unit 262, has changed from the kind TX to a kind TY right after the above-described insertion control is performed, the insertion control unit 263 is configured to select insertion control information CJY including a control content corresponding to an insertion shape of the kind TY from among the plurality of pieces of insertion control information stored in the storage medium 20M in advance.

Furthermore, according to the series of pieces of processing in FIG. 14, when having detected that the kind of the insertion shape of the insertion portion 11, which is indicated as a classification result obtained by the insertion shape classification unit 262, has not changed from the kind TX right after the above-described insertion control is performed, the insertion control unit 263 is configured to determine whether it is needed to change the control content included in the insertion control information CJX.

Note that, in the series of pieces of processing in FIG. 14, for example, the insertion control unit 263 skips the processing at step S1 in FIG. 14 and performs the processing at step S2 in FIG. 14 or later when having acquired a detection result that the kind of the insertion shape of the insertion portion 11 has changed at the processing at step S5 in FIG. 14 and having detected that insertion control information corresponding to the kind of the insertion shape before the change can be continuously used for the kind of the insertion shape after the change.

Subsequently, effects of the present modification will be described below. Note that the description below will be made on a specific example in which the above-described control in FIG. 14 is applied to the insertion portion 11 inserted into the intestinal canal of the large intestine through the anus. A control content (such as control amount) included in each piece of insertion control information described below is an example when the insertion portion 11 is inserted into the intestinal canal of the large intestine, and thus may be changed as appropriate in accordance with an application site of the endoscope 10 or the like.

A user such as a surgeon connects components of the endoscope system 1 and powers on the endoscope system 1, and then disposes the insertion portion 11 so that, for example, the distal end portion 12 is positioned near the anus or rectum of a subject.

According to an operation by the user as described above, an object is irradiated with illumination light supplied from the light source unit 210, image pickup of the object irradiated with the illumination light is performed by the image pickup unit 110, and an endoscope image obtained through the image pickup of the object is outputted from the image processing unit 220 to the display control unit 250 and the system control unit 260.

In addition, according to an operation by the user as described above, a coil drive signal is supplied from the coil drive signal generation unit 230, a magnetic field is generated by each of the plurality of source coils 18 in accordance with the coil drive signal, insertion shape information obtained by detecting the magnetic field is outputted from the insertion shape information acquisition unit 320 to the system control unit 260, and an insertion shape image in accordance with the insertion shape information is generated by the insertion shape image generation unit 261.

In addition, according to an operation by the user as described above, external force information indicating the magnitude and direction of external force at the position of each of the plurality of source coils 18 is outputted from the external force information acquisition device 40 to the system control unit 260.

In a state in which the insertion portion 11 is disposed as described above, for example, the user turns on the automatic insertion switch of the input device 50 to provide an instruction for starting insertion control of the insertion portion 11 by the main body device 20.

When having detected the instruction for starting insertion control of the insertion portion 11, the classification result recording unit 264 starts operation for recording classification results on which one insertion control of an insertion operation of the insertion portion 11 is based in time series each time the insertion control unit 263 performs the insertion control for the endoscope function control unit 240.

Note that, when such operation of the classification result recording unit 264 is performed, for example, graphs obtained by replacing, with “the number of controls”, “time” on the horizontal axis in the graphs illustrated in FIGS. 13A to 13C can be produced as graphs indicating temporal transition of the kind of the insertion shape of the insertion portion 11 along with control by the insertion control unit 263.

For example, when the insertion shape image SGA as illustrated in FIG. 3 is generated by the insertion shape image generation unit 261, the insertion shape classification unit 262 acquires a classification result that the insertion shape of the insertion portion 11 is classified as the kind TA.

When having detected that the kind of the insertion shape of the insertion portion 11 is the kind TA based on the classification result obtained by the insertion shape classification unit 262, the insertion control unit 263 performs processing for selecting and reading insertion control information CJA corresponding to the kind TA from among a plurality of pieces of insertion control information stored in the storage medium 20M in advance (equivalent to step S1 in FIG. 14).

The above-described insertion control information CJA includes the information related to a method for producing a state in which the insertion portion 11 can move forward. The above-described insertion control information CJA also includes, as information indicating control content for one time, for example, information that the insertion portion 11 is moved forward under conditions of a forward movement amount of 50 mm, a forward movement speed of 30 mm per second, and a propulsive force of 2.0 N or smaller.

When having detected that the insertion control information CJA includes the information related to a method for producing a state in which the insertion portion 11 can move forward (equivalent to YES at S2), the insertion control unit 263 performs, based on an endoscope image outputted from the image processing unit 220, processing for detecting a position of a lumen region in the endoscope image.

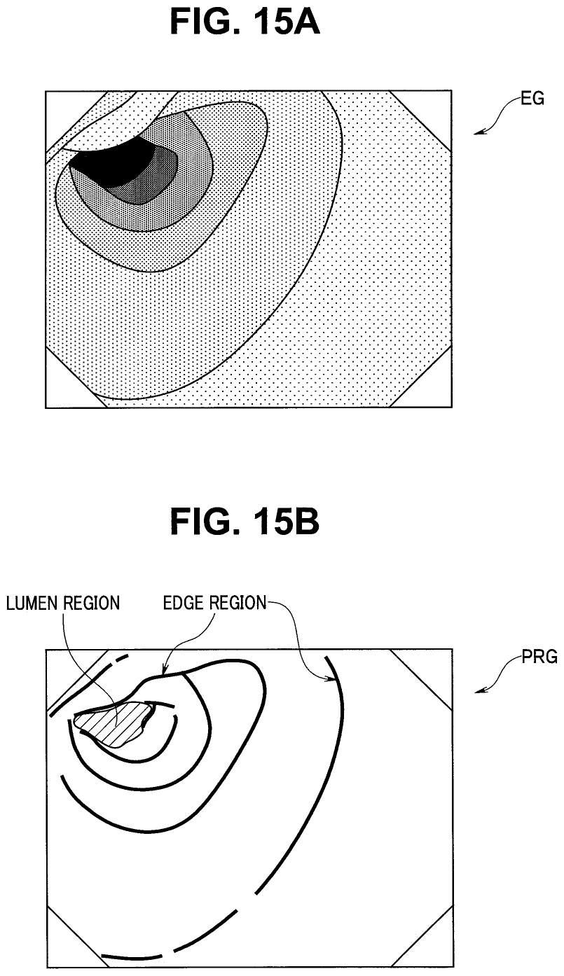

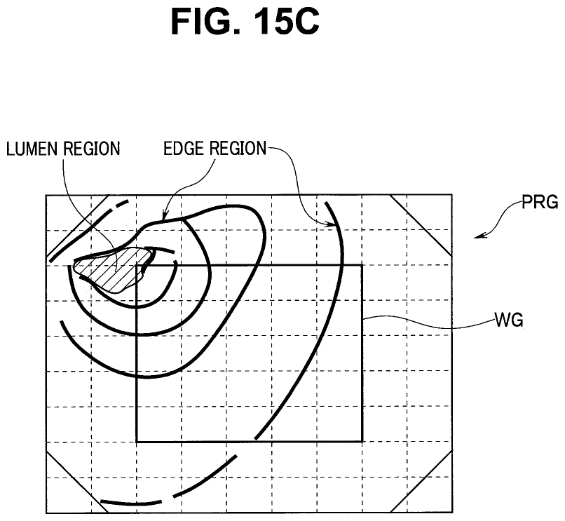

Specifically, the insertion control unit 263 performs, for example, processing that acquires a processing result image PRG as illustrated in FIG. 15B by inputting an endoscope image EG as illustrated in FIG. 15A to the learning-completed classifier CLQ including a fully convolutional neural network (FCN). FIG. 15A is a diagram illustrating an example of an endoscope image generated in the endoscope system according to the embodiment. FIG. 15B is a diagram illustrating an example of a processing result image obtained when processing for detecting a position of a lumen region is performed on the endoscope image in FIG. 15A.

At production of the above-described classifier CLQ, for example, machine learning is performed by using teacher data including an endoscope image same as an endoscope image generated by the image processing unit 220 and a label indicating to which, of an edge, a lumen, and another part, each pixel included in the endoscope image belongs.

Thus, with the above-described classifier CLQ, for example, the processing result image PRG with which it is possible to specify a position of any edge region and a position of any lumen region in an endoscope image generated by the image processing unit 220 can be acquired as output data by acquiring multi-dimensional data such as a pixel value of each pixel included in the endoscope image and inputting the multi-dimensional data as input data to the input layer of the neural network. Accordingly, the processing result image obtained through the above-described processing using the classifier CLQ includes a region division result corresponding to semantic segmentation.

Note that, according to the present modification, for example, when having determined that it is difficult to specify a lumen region with the processing result image PRG, the insertion control unit 263 may generate an insertion control signal for operating the AWS control unit 243 to perform air-water feeding and/or suction by the AWS mechanism 143 and may output the insertion control signal to the endoscope function control unit 240.

The insertion control unit 263 generates, based on a control content included in the insertion control information CJA, an insertion control signal for performing operation to place a lumen region detected from an endoscope image outputted from the image processing unit 220 in a predetermined region including a central portion in the endoscope image, and outputs the insertion control signal to the endoscope function control unit 240 (equivalent to step S3 in FIG. 14).

Specifically, for example, based on the control content included in the insertion control information CJA, the insertion control unit 263 divides the processing result image PRG in FIG. 15B into 9 x 9 regions as illustrated in FIG. 15C, generates an insertion control signal for adjusting the orientation of the distal end portion 12 and/or a rotational angle of the insertion portion 11 so that an entire range or substantially entire range of the lumen region included in the processing result image PRG is positioned in 5×5 regions including the central portion of the processing result image PRG (inside the frame WG in FIG. 15C), and outputs the insertion control signal to the endoscope function control unit 240.

Then, at least one of control by the bending control unit 242 for bending the bending portion 13 through the bending mechanism 142 or control by the rotation control unit 244 for rotating the insertion portion 11 through the rotation mechanism 144 is performed along with such control by the insertion control unit 263. In addition, the insertion portion 11 enters a state in which the insertion portion 11 can move forward along with the control by the insertion control unit 263 as described above. FIG. 15C is a diagram for description of control performed when the processing result image in FIG. 15B is obtained.

The insertion control unit 263 repeatedly performs control on the endoscope function control unit 240 until the lumen region is positioned in the predetermined region including the central portion in the endoscope image outputted from the image processing unit 220.

When having detected that the lumen region is positioned in the predetermined region including the central portion in the endoscope image outputted from the image processing unit 220, the insertion control unit 263 generates, based on external force information outputted from the external force information acquisition device 40, an insertion control signal for performing one control in accordance with the control content included in the insertion control information CJA, and outputs the insertion control signal to the endoscope function control unit 240 (equivalent to step S4 in FIG. 14). Then, in accordance with such control by the insertion control unit 263, the forward-backward movement control unit 241 performs control for moving forward the insertion portion 11 through the forward-backward movement mechanism 141.

The insertion control unit 263 detects, based on a classification result obtained by the insertion shape classification unit 262, whether the kind of the insertion shape of the insertion portion 11 has changed from the kind TA in accordance with one control based on the control content included in the insertion control information CJA (equivalent to step S5 in FIG. 14).

When having acquired a detection result that the kind of the insertion shape of the insertion portion 11 has not changed from the kind TA (equivalent to NO at S5), the insertion control unit 263 determines whether it is needed to further change the control content included in the insertion control information CJA (equivalent to step S6 in FIG. 14).

For example, when having detected that external force applied when the insertion portion 11 is moved forward is equal to or smaller than 2.0 N based on external force information outputted from the external force information acquisition device 40, the insertion control unit 263 acquires a determination result that it is not needed to change the control content included in the insertion control information CJA (equivalent to NO at S6).

When having acquired a determination result that it is not needed to change the control content included in the insertion control information CJA, the insertion control unit 263 performs control for producing a state in which the insertion portion 11 can move forward, and then generates an insertion control signal for performing one control in accordance with the control content included in the insertion control information CJA, and outputs the insertion control signal to the endoscope function control unit 240 (equivalent to steps S2, S3, and S4 in FIG. 14).

For example, when having detected that external force applied when the insertion portion 11 is moved forward exceeds 2.0 N based on external force information outputted from the external force information acquisition device 40, the insertion control unit 263 acquires a determination result that it is needed to change the control content included in the insertion control information CJA (equivalent to YES at S6).

When having acquired a determination result that it is needed to change the control content included in the insertion control information CJA, the insertion control unit 263 sets a changed control content that is obtained by adding jiggling to the control content included in the insertion control information CJA (equivalent to step S7 in FIG. 14). Then, the insertion control unit 263 performs control for producing a state in which the insertion portion 11 can move forward, and then generates an insertion control signal for performing one control in accordance with the above-described changed control content (including jiggling) and outputs the insertion control signal to the endoscope function control unit 240 (equivalent to steps S2, S3, and S4 in FIG. 14).

Note that the above-described jiggling is performed as an operation for moving forward and backward the insertion portion 11 inserted into the intestinal canal little by little at manual insertion of the insertion portion 11. The above-described jiggling is also performed as an operation to remove or mitigate phenomena, such as deflection of the insertion portion 11 inserted into the intestinal canal, friction that occurs between the intestinal canal and the insertion portion 11, and catch of the insertion portion 11 in the intestinal canal, which would interfere with manual insertion of the insertion portion 11 in the large intestine. Thus, for example, an operation corresponding to the above-described jiggling can be achieved by generating, through the forward-backward movement control unit 241, a forward-backward movement control signal for performing control to repeatedly move forward and backward the insertion portion 11 little by little a certain number of times, and by outputting the forward-backward movement control signal to the forward-backward movement mechanism 141.

When jiggling is added to the control content included in the insertion control information CJA, control to jiggle the insertion portion 11 and move forward the insertion portion 11 in accordance with the control content included in the insertion control information CJA is performed as one control by the insertion control unit 263.

Note that, according to the present modification, for example, when it is detected that the kind of the insertion shape of the insertion portion 11 has not changed from the kind TA although a predetermined number of controls are performed in a state in which jiggling is added to the control content included in the insertion control information CJA, control to move backward the insertion portion 11 by a certain amount and then jiggle and move forward the insertion portion 11 may be performed by the insertion control unit 263.

For example, when the insertion shape image SGB1 as illustrated in FIG. 4A or the insertion shape image SGB2 as illustrated in FIG. 4B is generated by the insertion shape image generation unit 261, the insertion shape classification unit 262 acquires a classification result that the insertion shape of the insertion portion 11 is classified as the kind TB.

When having acquired a detection result that the kind of the insertion shape of the insertion portion 11 has changed from the kind TA to the kind TB (equivalent to YES at S5 and NO at S8), the insertion control unit 263 performs processing for selecting and reading insertion control information CJB corresponding to the kind TB from among a plurality of pieces of insertion control information stored in the storage medium 20M in advance (equivalent to step S1 in FIG. 14).

The above-described insertion control information CJB includes the information related to a method for producing a state in which the insertion portion 11 can move forward. The above-described insertion control information CJB also includes, as information indicating control content for one time, for example, information that the insertion portion 11 is moved forward under conditions of a forward movement amount of 20 mm, a forward movement speed of 10 mm per second, and a propulsive force of 3.0 N or smaller after it is detected that external force applied when the insertion portion 11 is moved forward by jiggling is equal to or smaller than 3.0 N.

When having detected that the insertion control information CJB includes the information related to a method for producing a state in which the insertion portion 11 can move forward (equivalent to YES at S2), the insertion control unit 263 performs, based on an endoscope image outputted from the image processing unit 220, processing for detecting a position of a lumen region in the endoscope image.

In addition, the insertion control unit 263 generates, based on a control content included in the insertion control information CJB, an insertion control signal for performing operation through which the lumen region detected by the above-described processing is positioned in a certain region including a central portion in the endoscope image, and outputs the insertion control signal to the endoscope function control unit 240 (equivalent to step S3 in FIG. 14).

Specifically, based on the control content included in the insertion control information CJB, the insertion control unit 263 acquires the processing result image PRG as exemplarily illustrated in FIG. 15B, divides the processing result image PRG into 9×9 regions, generates an insertion control signal for adjusting the orientation of the distal end portion 12 and/or the rotational angle of the insertion portion 11 so that an entire range or substantially entire range of the lumen region included in the processing result image PRG is positioned in 7×7 regions including the central portion of the processing result image PRG, and outputs the insertion control signal to the endoscope function control unit 240.

Then, at least one of control by the bending control unit 242 for bending the bending portion 13 through the bending mechanism 142 or control by the rotation control unit 244 for rotating the insertion portion 11 through the rotation mechanism 144 is performed along with such control by the insertion control unit 263. In addition, the insertion portion 11 enters a state in which the insertion portion 11 can move forward along with the control by the insertion control unit 263 as described above.

The insertion control unit 263 repeatedly performs control on the endoscope function control unit 240 until the lumen region is positioned in the certain region including the central portion in the endoscope image outputted from the image processing unit 220. When having detected that the lumen region is positioned in the certain region including the central portion in the endoscope image outputted from the image processing unit 220, the insertion control unit 263 generates, based on external force information outputted from the external force information acquisition device 40, an insertion control signal for performing one control in accordance with the control content included in the insertion control information CJB, and outputs the insertion control signal to the endoscope function control unit 240 (equivalent to step S4 in FIG. 14).

Then, control for jiggling the insertion portion 11 through the forward-backward movement mechanism 141 and control for moving forward the insertion portion 11 through the forward-backward movement mechanism 141 are sequentially performed by the forward-backward movement control unit 241 in accordance with such control by the insertion control unit 263.

The insertion control unit 263 detects, based on a classification result obtained by the insertion shape classification unit 262, whether the kind of the insertion shape of the insertion portion 11 has changed from the kind TB in accordance with one control based on the control content included in the insertion control information CJB (equivalent to step S5 in FIG. 14).

When having acquired a detection result that the kind of the insertion shape of the insertion portion 11 has not changed from the kind TB (equivalent to NO at S5), the insertion control unit 263 determines whether it is needed to further change the control content included in the insertion control information CJB (equivalent to step S6 in FIG. 14).

In a duration in which control is performed in accordance with the insertion control information CJB read from the storage medium 20M, the insertion control unit 263 acquires a determination result that it is not needed to change the control content included in the insertion control information CJB (equivalent to NO at S6).

When having acquired a determination result that it is not needed to change the control content included in the insertion control information CJB, the insertion control unit 263 performs control for producing a state in which the insertion portion 11 can move forward, and then generates an insertion control signal for performing one control in accordance with the control content included in the insertion control information CJB, and outputs the insertion control signal to the endoscope function control unit 240 (equivalent to steps S2, S3, and S4 in FIG. 14).

For example, when the insertion shape image SGC1 as illustrated in FIG. 5A or the insertion shape image SGC2 as illustrated in FIG. 5B is generated by the insertion shape image generation unit 261, the insertion shape classification unit 262 acquires a classification result that the insertion shape of the insertion portion 11 is classified as the kind TC.

When having acquired a detection result that the kind of the insertion shape of the insertion portion 11 has changed from the kind TB to the kind TC (equivalent to YES at S5 and NO at S8), the insertion control unit 263 performs control in accordance with the insertion control information CJB read from the storage medium 20M again. In other words, when having detected that the kind of the insertion shape of the insertion portion 11 has changed from the kind TB to the kind TC, the insertion control unit 263 determines that the insertion control information CJB corresponding to the kind TB can be continuously used, skips the processing at step S1 in FIG. 14, and performs the processing at step S2 in FIG. 14 or later.

For example, when the insertion shape image SGD1 as illustrated in FIG. 6A or the insertion shape image SGD2 as illustrated in FIG. 6B is generated by the insertion shape image generation unit 261, the insertion shape classification unit 262 acquires a classification result that the insertion shape of the insertion portion 11 is classified as the kind TD.

When having acquired a detection result that the kind of the insertion shape of the insertion portion 11 has changed from the kind TC to the kind TD (equivalent to YES at S5 and NO at S8), the insertion control unit 263 performs processing for selecting and reading insertion control information CJD corresponding to the kind TD from among a plurality of pieces of insertion control information stored in the storage medium 20M in advance (equivalent to step S1 in FIG. 14).

The above-described insertion control information CJD includes the information related to a method for producing a state in which the insertion portion 11 can move forward. The above-described insertion control information CJD also includes, as information indicating control content for one time, for example, information that the insertion portion 11 is moved forward under conditions of a forward movement amount of 20 mm, a forward movement speed of 20 mm per second, and a propulsive force of 2.5 N or smaller after it is detected that external force applied when the insertion portion 11 is moved forward by jiggling is equal to or smaller than 2.5 N.

When having detected that the insertion control information CJD includes the information related to a method for producing a state in which the insertion portion 11 can move forward (equivalent to YES at S2), the insertion control unit 263 performs, based on an endoscope image outputted from the image processing unit 220, processing for detecting a position of a lumen region in the endoscope image.

In addition, the insertion control unit 263 generates, based on a control content included in the insertion control information CJD, an insertion control signal for performing operation through which the lumen region detected by the above-described processing is positioned in a certain region including a central portion in the endoscope image, and outputs the insertion control signal to the endoscope function control unit 240 (equivalent to step S3 in FIG. 14).

Specifically, based on the control content included in the insertion control information CJD, the insertion control unit 263 acquires the processing result image PRG as exemplarily illustrated in FIG. 15B, divides the processing result image PRG into 9×9 regions, generates an insertion control signal for adjusting the orientation of the distal end portion 12 and/or the rotational angle of the insertion portion 11 so that an entire range or substantially entire range of the lumen region included in the processing result image PRG is positioned in 5×5 regions including the central portion in the processing result image PRG, and outputs the insertion control signal to the endoscope function control unit 240.

For example, when having detected that the lumen region is not positioned in the above-described 5×5 regions although the insertion portion 11 is rotated in a state in which a bending angle of the bending portion 13 has reached a maximum value, the insertion control unit 263 generates an insertion control signal for adjusting the orientation of the distal end portion 12 and/or the rotational angle of the insertion portion 11 so that an entire range or substantially entire range of the lumen region included in the processing result image PRG is positioned in 7×7 regions including the central portion in the processing result image PRG, and outputs the insertion control signal to the endoscope function control unit 240.

Then, at least one of control by the bending control unit 242 for bending the bending portion 13 through the bending mechanism 142 or control by the rotation control unit 244 for rotating the insertion portion 11 through the rotation mechanism 144 is performed along with such control by the insertion control unit 263. In addition, the insertion portion 11 enters a state in which the insertion portion 11 can move forward along with the control by the insertion control unit 263 as described above.

The insertion control unit 263 repeatedly performs control on the endoscope function control unit 240 until the lumen region is positioned in the certain region including the central portion in the endoscope image outputted from the image processing unit 220.

When having detected that the lumen region is positioned in the certain region including the central portion in the endoscope image outputted from the image processing unit 220, the insertion control unit 263 generates, based on external force information outputted from the external force information acquisition device 40, an insertion control signal for performing one control in accordance with the control content included in the insertion control information CJD, and outputs the insertion control signal to the endoscope function control unit 240 (equivalent to step S4 in FIG. 14).

Then, control for jiggling the insertion portion 11 through the forward-backward movement mechanism 141 and control for moving forward the insertion portion 11 through the forward-backward movement mechanism 141 are sequentially performed by the forward-backward movement control unit 241 in accordance with such control by the insertion control unit 263.

The insertion control unit 263 detects, based on a classification result obtained by the insertion shape classification unit 262, whether the kind of the insertion shape of the insertion portion 11 has changed from the kind TD in accordance with one control based on the control content included in the insertion control information CJD (equivalent to step S5 in FIG. 14).

When having acquired a detection result that the kind of the insertion shape of the insertion portion 11 has not changed from the kind TD (equivalent to NO at S5), the insertion control unit 263 determines whether it is needed to further change the control content included in the insertion control information CJD (equivalent to step S6 in FIG. 14).

In a duration in which control is performed in accordance with the insertion control information CJD read from the storage medium 20M, the insertion control unit 263 acquires a determination result that it is not needed to change the control content included in the insertion control information CJD (equivalent to NO at S6).

When having acquired a determination result that it is not needed to change the control content included in the insertion control information CJD, the insertion control unit 263 performs control for producing a state in which the insertion portion 11 can move forward, and then generates an insertion control signal for performing one control in accordance with the control content included in the insertion control information CJD and outputs the insertion control signal to the endoscope function control unit 240 (equivalent to steps S2, S3, and S4 in FIG. 14).

For example, when the insertion shape image SGE1 as illustrated in FIG. 7A or the insertion shape image SGE2 as illustrated in FIG. 7B is generated by the insertion shape image generation unit 261, the insertion shape classification unit 262 acquires a classification result that the insertion shape of the insertion portion 11 is classified as the kind TE.

When having acquired a detection result that the kind of the insertion shape of the insertion portion 11 has changed from the kind TD to the kind TE (equivalent to YES at S5 and NO at S8), the insertion control unit 263 performs processing for selecting and reading insertion control information CJE corresponding to the kind TE from among a plurality of pieces of insertion control information stored in the storage medium 20M in advance (equivalent to step S1 in FIG. 14).

The above-described insertion control information CJE includes information related to a method for disentangling a loop shape formed by the insertion portion 11. The above-described insertion control information CJE also includes, as information indicating control content for one time, for example, information indicating a backward movement amount BLA by which the insertion portion 11 is moved backward and information indicating a backward movement speed BVA at which the insertion portion 11 is moved backward.

When having detected that the insertion control information CJE includes information related to a method for disentangling a loop shape formed by the insertion portion 11 (equivalent to NO at S2), the insertion control unit 263 generates an insertion control signal for performing one control in accordance with the backward movement amount BLA and the backward movement speed BVA included in the insertion control information CJE, and outputs the insertion control signal to the endoscope function control unit 240 (equivalent to step S4 in FIG. 14). Then, control for moving backward the insertion portion 11 through the forward-backward movement mechanism 141 is performed by the forward-backward movement control unit 241 in accordance with such control by the insertion control unit 263.

The insertion control unit 263 detects, based on a classification result obtained by the insertion shape classification unit 262, whether the kind of the insertion shape of the insertion portion 11 has changed from the kind TE in accordance with one control based on a control content included in the insertion control information CJE (equivalent to step S5 in FIG. 14).

When having acquired a detection result that the kind of the insertion shape of the insertion portion 11 has not changed from the kind TE (equivalent to NO at S5), the insertion control unit 263 determines whether it is needed to further change the control content included in the insertion control information CJE (equivalent to step S6 in FIG. 14).

For example, when having detected that disentanglement (loosening) of an α loop is in progress based on an endoscope image outputted from the image processing unit 220, external force information outputted from the external force information acquisition device 40, and an insertion shape image generated by the insertion shape image generation unit 261, the insertion control unit 263 acquires a determination result that it is not needed to change the control content included in the insertion control information CJE (equivalent to NO at S6).

When having acquired a determination result that it is not needed to change the control content included in the insertion control information CJE, the insertion control unit 263 generates an insertion control signal for performing one control in accordance with the control content included in the insertion control information CJE and outputs the insertion control signal to the endoscope function control unit 240 (equivalent to steps S2 and S4 in FIG. 14).

For example, when having detected that disentanglement (loosening) of an a loop is not in progress based on an endoscope image outputted from the image processing unit 220, external force information outputted from the external force information acquisition device 40, and an insertion shape image generated by the insertion shape image generation unit 261, the insertion control unit 263 acquires a determination result that it is needed to change the control content included in the insertion control information CJE (equivalent to YES at S6).

When having acquired a determination result that it is needed to change the control content included in the insertion control information CJE, the insertion control unit 263 sets, for example, a changed control content in which at least one parameter of the backward movement amount BLA or the backward movement speed BVA included in the insertion control information CJE is changed (equivalent to step S7 in FIG. 14).

Alternatively, when having acquired a determination result that it is needed to change the control content included in the insertion control information CJE, the insertion control unit 263 sets, for example, a changed control content that is obtained by adding a control content of another kind related to an insertion operation of the insertion portion 11 to the control content included in the insertion control information CJE (equivalent to step S7 in FIG. 14).

Then, the insertion control unit 263 generates an insertion control signal for performing one control in accordance with the above-described changed control content and outputs the insertion control signal to the endoscope function control unit 240 (equivalent to steps S2 and S4 in FIGS. 14).

Note that the insertion control unit 263 of the present modification is not limited to a configuration of starting control in accordance with the insertion control information CJE right after having acquired a detection result that the kind of the insertion shape of the insertion portion 11 has changed from the kind TD to the kind TE, but may be configured to start control in accordance with the insertion control information CJE, for example, when having continuously acquired, for one minute, a detection result that the kind of the insertion shape of the insertion portion 11 has changed from the kind TD to the kind TE.

For example, when the insertion shape image SGF1 as illustrated in FIG. 8A or the insertion shape image SGF2 as illustrated in FIG. 8B is generated by the insertion shape image generation unit 261, the insertion shape classification unit 262 acquires a classification result that the insertion shape of the insertion portion 11 is classified as the kind TF.

When having acquired a detection result that the kind of the insertion shape of the insertion portion 11 has changed from the kind TE to the kind TF (equivalent to YES at S5 and NO at S8), the insertion control unit 263 performs processing for selecting and reading insertion control information CJF corresponding to the kind TF from among a plurality of pieces of insertion control information stored in the storage medium 20M in advance (equivalent to step S1 in FIG. 14).

The above-described insertion control information CJF includes information related to a method for disentangling a loop shape formed by the insertion portion 11. The above-described insertion control information CJF also includes, as information indicating control content for one time, for example, information indicating a rotational angle BAA by which the insertion portion 11 is rotated rightward about the insertion axis (longitudinal axis).

When having detected that the insertion control information CJF includes information related to a method for disentangling a loop shape formed by the insertion portion 11 (equivalent to NO at S2), the insertion control unit 263 generates an insertion control signal for performing one control in accordance with the rotational angle BAA included in the insertion control information CJF and outputs the insertion control signal to the endoscope function control unit 240 (equivalent to step S4 in FIG. 14). Then, control for rotating the insertion portion 11 rightward about the insertion axis (longitudinal axis) through the rotation mechanism 144 is performed by the rotation control unit 244 in accordance with such control by the insertion control unit 263.

The insertion control unit 263 detects, based on a classification result obtained by the insertion shape classification unit 262, whether the kind of the insertion shape of the insertion portion 11 has changed from the kind TF in accordance with one control based on a control content included in the insertion control information CJF (equivalent to step S5 in FIG. 14).

When having acquired a detection result that the kind of the insertion shape of the insertion portion 11 has not changed from the kind TF (equivalent to NO at S5), the insertion control unit 263 determines whether it is needed to further change the control content included in the insertion control information CJF (equivalent to step S6 in FIG. 14).

For example, when having detected that disentanglement (loosening) of an a loop is in progress based on an endoscope image outputted from the image processing unit 220, external force information outputted from the external force information acquisition device 40, and an insertion shape image generated by the insertion shape image generation unit 261, the insertion control unit 263 acquires a determination result that it is not needed to change the control content included in the insertion control information CJF (equivalent to NO at S6).

When having acquired a determination result that it is not needed to change the control content included in the insertion control information CJF, the insertion control unit 263 generates an insertion control signal for performing one control in accordance with the control content included in the insertion control information CJF and outputs the insertion control signal to the endoscope function control unit 240 (equivalent to steps S2 and S4 in FIG. 14).

For example, when having detected that disentanglement (loosening) of an a loop is not in progress based on an endoscope image outputted from the image processing unit 220, external force information outputted from the external force information acquisition device 40, and an insertion shape image generated by the insertion shape image generation unit 261, the insertion control unit 263 acquires a determination result that it is needed to change the control content included in the insertion control information CJF (equivalent to YES at S6).

When having acquired a determination result that it is needed to change the control content included in the insertion control information CJF, the insertion control unit 263 sets, for example, a changed control content in which operation to move backward the insertion portion 11 by a certain amount is additionally performed before the insertion portion 11 is rotated by the rotational angle BAA (equivalent to step S7 in FIG. 14).

Then, the insertion control unit 263 generates an insertion control signal for performing one control in accordance with the above-described changed control content and outputs the insertion control signal to the endoscope function control unit 240 (equivalent to steps S2 and S4 in FIG. 14).

Note that, according to the present modification, for example, when it is detected that disentanglement (loosening) of an α loop is not in progress although a certain number of controls are performed in a state in which the control content included in the insertion control information CJF is changed, control to move forward the insertion portion 11 in a state in which the α loop is formed may be performed by the insertion control unit 263.

For example, when the insertion shape image SGG1 as illustrated in FIG. 9A or the insertion shape image SGG2 as illustrated in FIG. 9B is generated by the insertion shape image generation unit 261, the insertion shape classification unit 262 acquires a classification result that the insertion shape of the insertion portion 11 is classified as the kind TG.

When having acquired a detection result that the kind of the insertion shape of the insertion portion 11 has changed from the kind TF to the kind TG (equivalent to YES at S5 and NO at S8), the insertion control unit 263 performs processing for selecting and reading insertion control information CJG corresponding to the kind TG from among a plurality of pieces of insertion control information stored in the storage medium 20M in advance (equivalent to step S1 in FIG. 14).

The above-described insertion control information CJG includes information related to a method for disentangling a loop shape formed by the insertion portion 11. The above-described insertion control information CJG also includes, as information indicating control content for one time, for example, information indicating a backward movement amount BLB by which the insertion portion 11 is moved backward, information indicating a backward movement speed BVB at which the insertion portion 11 is moved backward, and information indicating a rotational angle BAB by which the insertion portion 11 is rotated rightward about the insertion axis (longitudinal axis). Note that the above-described backward movement speed BVB may be set to, for example, a speed of 15 mm per second approximately.

When having detected that the insertion control information CJG includes information related to a method for disentangling a loop shape formed by the insertion portion 11 (equivalent to NO at S2), the insertion control unit 263 generates an insertion control signal for performing one control in accordance with the backward movement amount BLB, the backward movement speed BVB, and the rotational angle BAB included in the insertion control information CJG and outputs the insertion control signal to the endoscope function control unit 240 (equivalent to step S4 in FIG. 14).

Then, control by the forward-backward movement control unit 241 for moving backward the insertion portion 11 through the forward-backward movement mechanism 141 and control by the rotation control unit 244 for rotating the insertion portion 11 rightward about the insertion axis (longitudinal axis) through the rotation mechanism 144 are simultaneously performed in accordance with such control by the insertion control unit 263.

The insertion control unit 263 detects, based on a classification result obtained by the insertion shape classification unit 262, whether the kind of the insertion shape of the insertion portion 11 has changed from the kind TG in accordance with one control based on a control content included in the insertion control information CJG (equivalent to step S5 in FIG. 14).

When having acquired a detection result that the kind of the insertion shape of the insertion portion 11 has not changed from the kind TG (equivalent to NO at S5), the insertion control unit 263 determines whether it is needed to further change the control content included in the insertion control information CJG (equivalent to step S6 in FIG. 14).

In a duration in which control is performed in accordance with the insertion control information CJG read from the storage medium 20M, the insertion control unit 263 acquires a determination result that it is not needed to change the control content included in the insertion control information CJG (equivalent to NO at S6).

When having acquired a determination result that it is not needed to change the control content included in the insertion control information CJG, the insertion control unit 263 generates an insertion control signal for performing one control in accordance with the control content included in the insertion control information CJG and outputs the insertion control signal to the endoscope function control unit 240 (equivalent to steps S2 and S4 in FIG. 14).

For example, when the insertion shape image SGH as illustrated in FIG. 10 is generated by the insertion shape image generation unit 261, the insertion shape classification unit 262 acquires a classification result that the insertion shape of the insertion portion 11 is classified as the kind TH.

When having acquired a detection result that the kind of the insertion shape of the insertion portion 11 has changed from the kind TG to the kind TH (equivalent to YES at S5 and NO at S8), the insertion control unit 263 performs processing for selecting and reading the insertion control information CJA corresponding to the kind TH from among a plurality of pieces of insertion control information stored in the storage medium 20M in advance (equivalent to step S1 in FIG. 14). Note that the above-described control in accordance with the control content included in the insertion control information CJA is applicable to control performed by the insertion control unit 263 when the kind of the insertion shape of the insertion portion 11 is the kind TH, and thus specific description is omitted.

In the present embodiment, when such a situation that the insertion portion 11 passes through the sigmoid colon without forming an α loop has occurred, the kind of the insertion shape of the insertion portion 11, which is indicated as a result of classification by the insertion shape classification unit 262, is maintained as the kind TA, and control in accordance with the control content included in the insertion control information CJA corresponding to the kind TA is continued by the insertion control unit 263. Thus, when having acquired a detection result that the kind of the insertion shape of the insertion portion 11 has changed from the kind TA to the kind TH (equivalent to YES at S5 and NO at S8), the insertion control unit 263 performs again control in accordance with the insertion control information CJA read from the storage medium 20M.

Specifically, when having detected that the kind of the insertion shape of the insertion portion 11 has changed from the kind TA to the kind TH, the insertion control unit 263 determines that the insertion control information CJA corresponding to the kind TA can be continuously used, skips the processing at step S1 in FIG. 14, and performs the processing at step S2 in FIG. 14 or later.

For example, when the insertion shape image SGI1 as illustrated in FIG. 11A or the insertion shape image SGI2 as illustrated in FIG. 11B is generated by the insertion shape image generation unit 261, the insertion shape classification unit 262 acquires a classification result that the insertion shape of the insertion portion 11 is classified as the kind TI.

When having acquired a detection result that the kind of the insertion shape of the insertion portion 11 has changed from the kind TH to the kind TI (equivalent to YES at S5 and NO at S8), the insertion control unit 263 performs again control in accordance with the insertion control information CJA read from the storage medium 20M.

Specifically, when having detected that the kind of the insertion shape of the insertion portion 11 has changed from the kind TH to the kind TI, the insertion control unit 263 determines that the insertion control information CJA corresponding to the kind TH can be continuously used, skips the processing at step S1 in FIG. 14, and performs the processing at step S2 in FIG. 14 or later.

For example, when the insertion shape image SGJ1 as illustrated in FIG. 12A or the insertion shape image SGJ2 as illustrated in FIG. 12B is generated by the insertion shape image generation unit 261, the insertion shape classification unit 262 acquires a classification result that the insertion shape of the insertion portion 11 is classified as the kind TJ.

When having acquired a detection result that the kind of the insertion shape of the insertion portion 11 has changed from the kind TI to the kind TJ (equivalent to YES at S5 and YES at S8), the insertion control unit 263 ends the series of controls on the endoscope function control unit 240.

For example, after having checked that the insertion shape of the insertion portion 11 inserted inside the subject has stopped changing based on an insertion shape image displayed on the display device 60, the user turns off the automatic insertion switch of the input device 50 to provide an instruction for stopping insertion control of the insertion portion 11 by the main body device 20.

When having detected the instruction for stopping insertion control of the insertion portion 11, the classification result recording unit 264 stops operation for recording results of classification by the insertion shape classification unit 262 in time series.

As described above, according to the present modification, the insertion shape classification unit 262 performs processing to obtain a classification result by classifying the kind of the insertion shape of the insertion portion 11 included in an insertion shape image generated by the insertion shape image generation unit 261, based on a viewpoint substantially equivalent to a viewpoint when an experienced and skilled person subjectively determines or evaluates whether an operation is successful and the like in an insertion operation of the insertion portion 11. Moreover, according to the above-described present modification, the insertion control unit 263 performs insertion control based on one piece of insertion control information in accordance with the kind of the insertion shape of the insertion portion 11, which is indicated as a classification result obtained by the insertion shape classification unit 262.

In addition, as described above, according to the present modification, the insertion control unit 263 performs operation to detect whether the kind of the insertion shape of the insertion portion 11 has changed each time one insertion control is performed based on one piece of insertion control information in accordance with the kind of the insertion shape of the insertion portion 11. Thus, according to the present modification, for example, it is possible to perform appropriate insertion control in accordance with an insertion situation of the insertion portion, such as individual difference in an internal state of a subject into which the insertion portion is inserted or temporal change of the insertion shape of the insertion portion inside the subject.

Note that the present modification is also applicable to, for example, control of disentanglement of a reversed α loop and an inverted α loop by replacing the rotational angles included in the insertion control information CJF and CJG with angles by which the insertion portion 11 is rotated leftward about the insertion axis (longitudinal axis).

The present modification is also applicable to, for example, control of disentanglement of various insertion shapes, such as a stick or a γ loop, which would interfere with insertion of the insertion portion 11 in the large intestine, by changing some control contents in the series of controls in FIG. 14.

### Second Embodiment

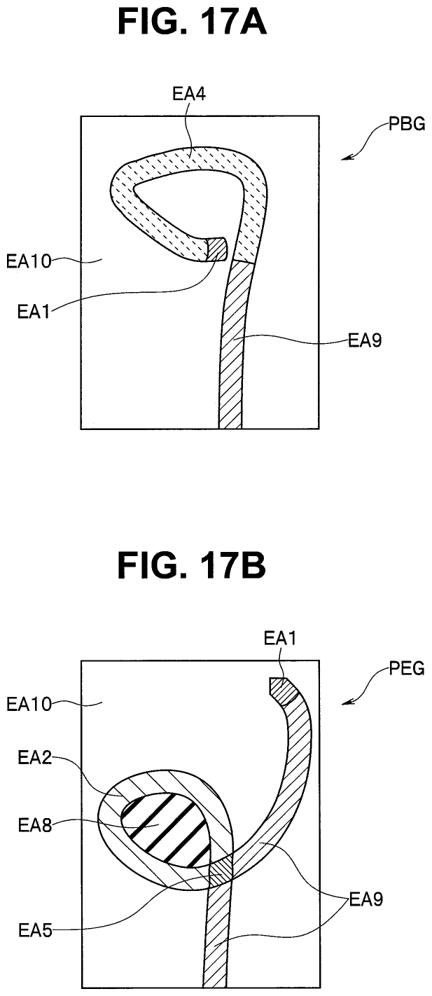

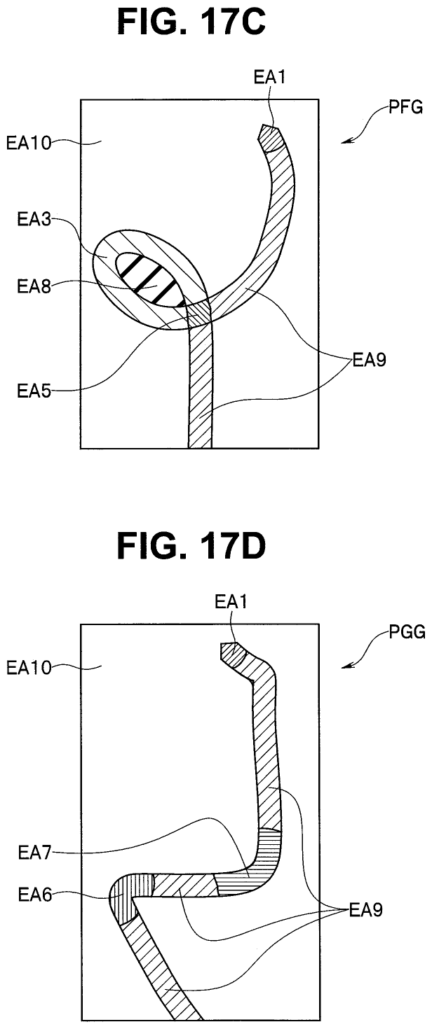

FIGS. 16 to 17D relate to a second embodiment.

Note that, in the present embodiment, detailed description related to any part having a configuration or the like same as a configuration or the like in the first embodiment is omitted as appropriate, and description will be mainly made on any part having a configuration or the like different from a configuration or the like in the first embodiment.

For example, as illustrated in FIG. 16, an endoscope system 1A includes the endoscope 10, a main body device 20A, the insertion shape detection device 30, the external force information acquisition device 40, the input device 50, and the display device 60. FIG. 16 is a block diagram for description of a specific configuration of the endoscope system according to the second embodiment.

The main body device 20A includes the processor 20P including one or more hardware components, and the storage medium 20M. As illustrated in FIG. 16, the main body device 20A also includes the light source unit 210, the image processing unit 220, the coil drive signal generation unit 230, the endoscope function control unit 240, the display control unit 250, and a system control unit 270.

The system control unit 270 is configured to generate and output a system control signal for performing operation in accordance with instructions and the like from the operation portion 16 and the input device 50. The system control unit 270 includes the insertion shape image generation unit 261, an insertion shape element extraction unit 272, an insertion control unit 273, and an extraction result recording unit 274.

The insertion shape element extraction unit 272 is configured to perform processing for obtaining an extraction result by extracting one or more constituent elements of the insertion shape of the insertion portion 11 from an insertion shape image generated by the insertion shape image generation unit 261.

<Specific Example of Configuration of Insertion Shape Element Extraction Unit 272>

A specific example of a configuration of the insertion shape element extraction unit 272 in the present embodiment will be described below.

The insertion shape element extraction unit 272 is configured to perform processing using a learning-completed classifier (for example, classifier CLR) including a fully convolutional neural network (FCN), thereby obtaining an extraction result that one or more constituent elements of the insertion shape of the insertion portion 11 are extracted from an insertion shape image generated by the insertion shape image generation unit 261.

At production of the above-described classifier CLR, machine learning is performed by using, for example, teacher data including an insertion shape image and a label, the insertion shape image being same as an insertion shape image generated by the insertion shape image generation unit 261, the label indicating to which constituent element each pixel included in the insertion shape image belongs among an endoscope distal end portion (hereinafter referred to as a constituent element E1), a relatively large closed loop (hereinafter referred to as a constituent element E2), a relatively small closed loop (hereinafter referred to as a constituent element E3), an open loop (hereinafter referred to as a constituent element E4), an intersection portion (hereinafter referred to as a constituent element E5) of a closed loop, an angled portion (hereinafter referred to as a constituent element E6) on the base end side of an N loop, an angled portion (hereinafter referred to as a constituent element E7) on the distal end side of an N loop, an inside (hereinafter referred to as a constituent element E8) of a closed loop, a part (hereinafter referred to as a constituent element E9) of the insertion portion of the endoscope other than the constituent elements E1 to E8, and a background (hereinafter referred to as a constituent element E10).

Presence, absence or the like of each of the above-described constituent elements E1 to E10 is determined by, for example, an experienced and skilled person having visually checked an insertion shape image used as teacher data.

A closed loop corresponding to the above-described constituent element E3 is defined as, for example, a loop having such a size that an experienced and skilled person attempts to disentangle the loop by performing a twisting operation on the insertion portion 11.

A closed loop corresponding to the above-described constituent element E2 is defined as a loop having a size larger than the size of the above-described constituent element E3.

The above-described constituent elements E1 to E8 are set as, for example, constituent elements for extracting, from one insertion shape image including the insertion shape of the insertion portion 11, any local region that affects determination of whether a manually or automatically performed insertion operation of the insertion portion 11 is successful and determination of whether it is needed to change an operation content.

Thus, with the above-described classifier CLR, for example, multi-dimensional data such as a pixel value of each pixel included in an insertion shape image generated by the insertion shape image generation unit 261 is acquired and inputted as input data to the input layer of the neural network, and accordingly, a processing result image illustrating a classification result that each pixel included in the insertion shape image is classified as any one of the above-described constituent elements E1 to E10 can be acquired as output data. Accordingly, the processing result image obtained through the above-described processing using the classifier CLR includes a region division result corresponding to semantic segmentation.

For example, when an insertion shape image including an insertion shape that would be classified as the kind TB through processing by the insertion shape classification unit 262 is generated by the insertion shape image generation unit 261, the insertion shape element extraction unit 272 acquires a processing result image PBG as illustrated in FIG. 17A by inputting the insertion shape image to the classifier CLR and performing processing. FIG. 17A is a diagram illustrating an example of an image illustrating an extraction result that constituent elements related to the insertion shape of the insertion portion are extracted from an insertion shape image generated in the endoscope system according to the second embodiment.

The processing result image PBG in FIG. 17A is generated as an image including a region division result that an insertion shape image generated by the insertion shape image generation unit 261 is divided into four regions of a region EA1 including a group of pixels classified as the constituent element E1, a region EA4 including a group of pixels classified as the constituent element E4, a region EA9 including a group of pixels classified as the constituent element E9, and a region EA10 including a group of pixels classified as the constituent element E10.

In other words, the processing result image PBG in FIG. 17A is acquired as an image illustrating an extraction result that three constituent elements corresponding to the constituent elements E1, E4, and E9 are extracted as constituent elements related to the insertion shape of the insertion portion 11 from an insertion shape image generated by the insertion shape image generation unit 261.

For example, when an insertion shape image including an insertion shape that is classified as the kind TE through processing by the insertion shape classification unit 262 is generated by the insertion shape image generation unit 261, the insertion shape element extraction unit 272 acquires a processing result image PEG as illustrated in FIG. 17B by inputting the insertion shape image to the classifier CLR and performing processing. FIG. 17B is a diagram illustrating an example of an image illustrating an extraction result that constituent elements related to the insertion shape of the insertion portion are extracted from an insertion shape image generated in the endoscope system according to the second embodiment.

The processing result image PEG in FIG. 17B is generated as an image including a region division result that an insertion shape image generated by the insertion shape image generation unit 261 is divided into six regions of a region EA1 including a group of pixels classified as the constituent element E1, a region EA2 including a group of pixels classified as the constituent element E2, a region EA5 including a group of pixels classified as the constituent element E5, a region EA8 including a group of pixels classified as the constituent element E8, a region EA9 including a group of pixels classified as the constituent element E9, and a region EA10 including a group of pixels classified as the constituent element E10.

In other words, the processing result image PEG in FIG. 17B is acquired as an image illustrating an extraction result that five constituent elements corresponding to the constituent elements E1, E2, E5, E8, and E9 are extracted as constituent elements related to the insertion shape of the insertion portion 11 from an insertion shape image generated by the insertion shape image generation unit 261.

For example, when an insertion shape image including an insertion shape that is classified as the kind TF through processing by the insertion shape classification unit 262 is generated by the insertion shape image generation unit 261, the insertion shape element extraction unit 272 acquires a processing result image PFG as illustrated in FIG. 17C by inputting the insertion shape image to the classifier CLR and performing processing. FIG. 17C is a diagram illustrating an example of an image illustrating an extraction result that constituent elements related to the insertion shape of the insertion portion are extracted from an insertion shape image generated in the endoscope system according to the second embodiment.

The processing result image PFG in FIG. 17C is generated as an image including a region division result that an insertion shape image generated by the insertion shape image generation unit 261 is divided into six regions of a region EA1 including a group of pixels classified as the constituent element E1, a region EA3 including a group of pixels classified as the constituent element E3, a region EA5 including a group of pixels classified as the constituent element E5, a region EA8 including a group of pixels classified as the constituent element E8, a region EA9 including a group of pixels classified as the constituent element E9, and a region EA10 including a group of pixels classified as the constituent element E10.

In other words, the processing result image PFG in FIG. 17C is acquired as an image illustrating an extraction result that five constituent elements corresponding to the constituent elements E1, E3, E5, E8, and E9 are extracted as constituent elements related to the insertion shape of the insertion portion 11 from an insertion shape image generated by the insertion shape image generation unit 261.

For example, when an insertion shape image including an insertion shape that is classified as the kind TG through processing by the insertion shape classification unit 262 is generated by the insertion shape image generation unit 261, the insertion shape element extraction unit 272 acquires a processing result image PGG as illustrated in FIG. 17D by inputting the insertion shape image to the classifier CLR and performing processing. FIG. 17D is a diagram illustrating an example of an image illustrating an extraction result that constituent elements related to the insertion shape of the insertion portion are extracted from an insertion shape image generated in the endoscope system according to the second embodiment.

The processing result image PGG in FIG. 17D is generated as an image including a region division result that an insertion shape image generated by the insertion shape image generation unit 261 is divided into five regions of a region EA1 including a group of pixels classified as the constituent element E1, a region EA6 including a group of pixels classified as the constituent element E6, a region EA7 including a group of pixels classified as the constituent element E7, a region EA9 including a group of pixels classified as the constituent element E9, and a region EA10 including a group of pixels classified as the constituent element E10.

In other words, the processing result image PGG in FIG. 17D is acquired as an image illustrating an extraction result that four constituent elements corresponding to the constituent elements E1, E6, E7, and E9 are extracted as constituent elements related to the insertion shape of the insertion portion 11 from an insertion shape image generated by the insertion shape image generation unit 261.

Specifically, the insertion shape element extraction unit 272 is configured to perform processing for obtaining an extraction result by extracting, as a constituent element related to the insertion shape of the insertion portion 11 from an insertion shape image generated by the insertion shape image generation unit 261, at least one of an endoscope distal end portion corresponding to the distal end portion 12, a loop portion corresponding to a loop-shaped part of the insertion portion 11, or an angled portion corresponding to an angled part of the insertion portion 11.

The insertion shape element extraction unit 272 is also configured to obtain an extraction result by extracting one or more constituent elements of the insertion shape of the insertion portion 11 inserted into the subject through processing using the classifier CLR produced by performing machine learning using teacher data including an insertion shape image illustrating the insertion shape of the insertion portion 11 and a label indicating a classification result that each pixel included in the insertion shape image is classified as one of a plurality of predetermined constituent elements.

The insertion control unit 273 is configured to generate, based on at least one of an endoscope image outputted from the image processing unit 220, external force information outputted from the external force information acquisition device 40, or an insertion shape image generated by the insertion shape image generation unit 261 and based on an extraction result obtained by the insertion shape element extraction unit 272, an insertion control signal including information for performing control of an insertion operation of the insertion portion 11, and is configured to output the insertion control signal to the endoscope function control unit 240.

Specifically, the insertion control unit 273 is configured to generate, based on at least one of an endoscope image outputted from the image processing unit 220, external force information outputted from the external force information acquisition device 40, or an insertion shape image generated by the insertion shape image generation unit 261 and based on an extraction result obtained by the insertion shape element extraction unit 272, an insertion control signal including information for performing, as control of an insertion operation of the insertion portion 11, for example, control of at least one of start of the insertion operation, continuation of the insertion operation, interruption of the insertion operation, resumption of the insertion operation, stop of the insertion operation, or completion of the insertion operation, and is configured to output the insertion control signal to the endoscope function control unit 240.

The insertion control unit 273 is also configured to generate, based on at least one of an endoscope image outputted from the image processing unit 220, external force information outputted from the external force information acquisition device 40, or an insertion shape image generated by the insertion shape image generation unit 261 and based on an extraction result obtained by the insertion shape element extraction unit 272, an insertion control signal including information for controlling at least one of an operation amount of an insertion operation of the insertion portion 11, operation speed of the insertion operation, or operation force of the insertion operation, and is configured to output the insertion control signal to the endoscope function control unit 240.

For example, the insertion control unit 273 of the present embodiment is configured to be able to set a control content in accordance with constituent elements of the current insertion shape of the insertion portion 11, which is indicated as an extraction result obtained by the insertion shape element extraction unit 272, based on at least one of an endoscope image outputted from the image processing unit 220, external force information outputted from the external force information acquisition device 40, or an insertion shape image generated by the insertion shape image generation unit 261, generate an insertion control signal including information for performing control of an insertion operation of the insertion portion 11 by using the set control content, and output the insertion control signal to the endoscope function control unit 240.

Thus, for example, the insertion control unit 273 can set an operation control group CGC including a control content for performing an insertion operation of the insertion portion 11 by executing alone a basic operation selected from among the basic operations achieved by respective functions of the endoscope 10 by setting a control content in accordance with constituent elements of the current insertion shape of the insertion portion 11, which is indicated as an extraction result obtained by the insertion shape element extraction unit 272, based on at least one of an endoscope image outputted from the image processing unit 220, external force information outputted from the external force information acquisition device 40, or an insertion shape image generated by the insertion shape image generation unit 261, and generate and output an insertion control signal including information of the set operation control group CGC.

The insertion control unit 273 can also set an operation control group CGD including a control content for performing an insertion operation of the insertion portion 11 by executing, for example, a combination of a plurality of basic operations selected from among the basic operations achieved by respective functions of the endoscope 10, by setting a control content in accordance with constituent elements of the current insertion shape of the insertion portion 11, which is indicated as an extraction result obtained by the insertion shape element extraction unit 272, based on at least one of an endoscope image outputted from the image processing unit 220, external force information outputted from the external force information acquisition device 40, or an insertion shape image generated by the insertion shape image generation unit 261, and generate and output an insertion control signal including information of the set operation control group CGD.

Note that the operation control group CGD is set as a control content for consecutively or simultaneously executing a plurality of basic operations selected from among the basic operations achieved by respective functions of the endoscope 10. In other words, the control content of the operation control group CGD is set as a more complicate control content than the control content of the operation control group CGC.

Specifically, the insertion control unit 273 is configured to perform, as control in accordance with constituent elements of the current insertion shape of the insertion portion 11, which is indicated as an extraction result obtained by the insertion shape element extraction unit 272, control based on any of the operation control group CGC including a control content for performing an insertion operation of the insertion portion 11 by executing alone a basic operation selected from among the basic operations achieved by respective functions of the endoscope 10 and the operation control group CGD including a control content for performing an insertion operation of the insertion portion 11 by executing a combination of a plurality of basic operations selected from among the basic operations achieved by respective functions of the endoscope 10.

The insertion control unit 273 is also configured to perform control of an insertion operation of the insertion portion based on at least one of an image obtained through image pickup of inside of the subject by the endoscope 10, information indicating magnitude of external force applied to the insertion portion 11, or information indicating the insertion shape of the insertion portion 11 and based on an extraction result obtained by the insertion shape element extraction unit 272.

The insertion control unit 273 is also configured to change a control content in accordance with temporal change of at least one constituent element included in an extraction result obtained by the insertion shape element extraction unit 272, based on at least one of an endoscope image outputted from the image processing unit 220, external force information outputted from the external force information acquisition device 40, or an insertion shape image generated by the insertion shape image generation unit 261.

The extraction result recording unit 274 is configured to be able to perform operation for recording extraction results obtained by the insertion shape element extraction unit 272 in time series.

In the present embodiment, at least some of functions of the main body device 20A may be achieved by the processor 20P. In addition, in the present embodiment, at least part of the main body device 20 may be configured as an individual electronic circuit or may be configured as a circuit block in an integrated circuit such as a field programmable gate array (FPGA).

In addition, a configuration according to the present embodiment may be modified as appropriate so that, for example, a computer reads a program for executing at least some of functions of the main body device 20A from the storage medium 20M such as a memory and performs operation in accordance with the read program.

Subsequently, effects of the present embodiment will be described below.

A user such as a surgeon connects components of the endoscope system 1A and powers on the endoscope system 1A, and then disposes the insertion portion 11 so that, for example, the distal end portion 12 is positioned near the anus or rectum of a subject.

According to an operation by the user as described above, an object is irradiated with illumination light supplied from the light source unit 210, image pickup of the object irradiated with the illumination light is performed by the image pickup unit 110, and an endoscope image obtained through the image pickup of the object is outputted from the image processing unit 220 to the display control unit 250 and the system control unit 270. In addition, according to an operation by the user as described above, a coil drive signal is supplied from the coil drive signal generation unit 230, a magnetic field is generated by each of the plurality of source coils 18 in accordance with the coil drive signal, insertion shape information obtained by detecting the magnetic field is outputted from the insertion shape information acquisition unit 320 to the system control unit 270, and an insertion shape image in accordance with the insertion shape information is generated by the insertion shape image generation unit 261.

In addition, according to an operation by the user as described above, external force information indicating the magnitude and direction of external force at the position of each of the plurality of source coils 18 is outputted from the external force information acquisition device 40 to the system control unit 270.

In a state in which the insertion portion 11 is disposed as described above, for example, the user turns on the automatic insertion switch of the input device 50 to provide an instruction for starting insertion control of the insertion portion 11 by the main body device 20A.

When having detected the instruction for starting insertion control of the insertion portion 11, the extraction result recording unit 274 starts, for example, operation for recording, in time series and at every constant time, extraction results obtained by the insertion shape element extraction unit 272.

The insertion control unit 273 sets a control content in accordance with constituent elements of the current insertion shape of the insertion portion 11, which is indicated as an extraction result obtained by the insertion shape element extraction unit 272, based on at least one of an endoscope image outputted from the image processing unit 220, external force information outputted from the external force information acquisition device 40, or an insertion shape image generated by the insertion shape image generation unit 261.

Specifically, when having detected that, for example, the constituent element E2 or E4 is included in an extraction result obtained by the insertion shape element extraction unit 272, the insertion control unit 273 generates and outputs an insertion control signal including information of the operation control group CGC including a control content set based on at least one of an endoscope image outputted from the image processing unit 220, external force information outputted from the external force information acquisition device 40, or an insertion shape image generated by the insertion shape image generation unit 261.

For example, when having detected that the constituent element E3 is included in an extraction result obtained by the insertion shape element extraction unit 272, the insertion control unit 273 generates and outputs an insertion control signal including information of the operation control group CGD including a control content set based on at least one of an endoscope image outputted from the image processing unit 220, external force information outputted from the external force information acquisition device 40, or an insertion shape image generated by the insertion shape image generation unit 261.

The insertion control unit 273 changes a control content in accordance with temporal change of at least one constituent element included in an extraction result obtained by the insertion shape element extraction unit 272, based on at least one of an endoscope image outputted from the image processing unit 220, external force information outputted from the external force information acquisition device 40, or an insertion shape image generated by the insertion shape image generation unit 261.

Specifically, the insertion control unit 273 performs, as processing for detecting temporal change of a position of the region EA1 included in a processing result image obtained by the insertion shape element extraction unit 272, for example, processing of generating a binarized image by binarizing the processing result image obtained by the insertion shape element extraction unit 272, processing for specifying a barycenter position of the region EA1 included in the binarized image, and processing of detecting temporal change of the barycenter position.

In addition, the insertion control unit 273 performs, as processing for detecting temporal change of area of the region EA8 included in a processing result image obtained by the insertion shape element extraction unit 272, for example, processing of generating a binarized image by binarizing the processing result image obtained by the insertion shape element extraction unit 272 and processing for detecting temporal change of number of pixels in the region EA8 included in the binarized image.

In addition, the insertion control unit 273 performs, as processing for detecting temporal change of a shape of the region EA8 included in the processing result image obtained by the insertion shape element extraction unit 272, for example, processing of generating a binarized image by binarizing the processing result image obtained by the insertion shape element extraction unit 272 and processing of detecting temporal change of a circularity degree of the region EA8 included in the binarized image.

In addition, the insertion control unit 273 performs, as processing for detecting temporal change of area of the region EA3 included in the processing result image obtained by the insertion shape element extraction unit 272, for example, processing of generating a binarized image by binarizing the processing result image obtained by the insertion shape element extraction unit 272 and processing of detecting temporal change of number of pixels in the region EA3 included in the binarized image.

In addition, the insertion control unit 273 performs, as processing for detecting temporal change of a length of the region EA3 included in the processing result image obtained by the insertion shape element extraction unit 272, for example, processing of generating a binarized image by binarizing the processing result image obtained by the insertion shape element extraction unit 272, processing for generating a line segment by thinning the region EA3 included in the binarized image and processing of detecting temporal change of number of pixels in the line segment.

Then, the insertion control unit 273 changes a control content in accordance with, for example, temporal change of at least one of the position of the region EA1, the area of the region EA8, the shape of the region EA8, the area of the region EA3, or the length of the region EA3, which are detected based on the processing result image obtained by the insertion shape element extraction unit 272, based on at least one of an endoscope image outputted from the image processing unit 220, external force information outputted from the external force information acquisition device 40, or an insertion shape image generated by the insertion shape image generation unit 261.

For example, after having checked that the insertion shape of the insertion portion 11 inserted inside the subject has stopped changing based on an insertion shape image displayed on the display device 60, the user turns off the automatic insertion switch of the input device 50 to provide an instruction for stopping insertion control of the insertion portion 11 by the main body device 20A.

When having detected the instruction for stopping insertion control of the insertion portion 11, the extraction result recording unit 274 stops operation for recording, in time series and at each constant time, extraction results obtained by the insertion shape element extraction unit 272.

As described above, according to the present embodiment, the insertion shape element extraction unit 272 performs processing to obtain an extraction result by extracting one or more constituent elements included in an insertion shape image generated by the insertion shape image generation unit 261, based on a viewpoint substantially equivalent to a viewpoint when an experienced and skilled person subjectively determines or evaluates whether an operation is successful and the like in an insertion operation of the insertion portion 11.

Moreover, according to the present embodiment, the insertion control unit 273 performs insertion control in accordance with one or more constituent elements included in an extraction result obtained by the insertion shape element extraction unit 272. Thus, according to the present embodiment, for example, it is possible to perform appropriate insertion control in accordance with an insertion situation of the insertion portion, such as individual difference in an internal state of the subject into which the insertion portion is inserted or temporal change of the insertion shape of the insertion portion inside the subject.

Note that, for example, the insertion control unit 273 of the present embodiment may be configured to perform control described in the modification of the first embodiment by using both a classification result obtained by the insertion shape classification unit 262 and an extraction result obtained by the insertion shape element extraction unit 272. Specific examples of processing and the like that can be performed in such a case will be listed below.

For example, when performing control of jiggling on the endoscope function control unit 240 in accordance with the control content included in the insertion control information CJB, the insertion control unit 273 acquires auxiliary information HJA that can be used to determine whether the jiggling is successful by detecting temporal change of the position of the region EA1 included in a processing result image obtained by the insertion shape element extraction unit 272. For example, during control in accordance with the control content included in the insertion control information CJB, the above-described auxiliary information HJA can be used to determine whether friction occurs between the insertion portion 11 and the intestinal canal and determine whether deflection occurs to the insertion portion 11.

For example, when having detected that the area of the region EA8 included in the processing result image obtained by the insertion shape element extraction unit 272 exceeds a predetermined value during control on the endoscope function control unit 240 in accordance with the control content included in the insertion control information CJD, the insertion control unit 273 performs control to move backward the insertion portion 11 by a predetermined backward movement amount and then move forward the insertion portion 11. With such control, it is possible to slightly loosen an α loop along with forward movement of the insertion portion 11.

For example, when having detected that the area or length of the region EA3 included in the processing result image obtained by the insertion shape element extraction unit 272 exceeds a predetermined value during control on the endoscope function control unit 240 in accordance with the control content included in the insertion control information CJD, the insertion control unit 273 performs control to move backward the insertion portion 11 by a predetermined backward movement amount and then move forward the insertion portion 11. With such control, it is possible to slightly loosen an α loop along with forward movement of the insertion portion 11.

For example, when having detected that the region EA4 in a processing result image obtained by the insertion shape element extraction unit 272 has changed to the region EA2 and the region EA5 has newly appeared in the processing result image, the insertion control unit 273 acquires a detection result that the insertion shape of the insertion portion 11 has changed from the kind TB to the kind TC.

For example, when having detected that the region EA2 in a processing result image obtained by the insertion shape element extraction unit 272 has changed to the region EA3, the insertion control unit 273 acquires a detection result that the insertion shape of the insertion portion 11 has changed from the kind TE to the kind TF.

For example, when performing control of disentanglement of an α loop formed by the insertion portion 11 on the endoscope function control unit 240, the insertion control unit 273 detects whether the insertion shape of the insertion portion 11 is any of the kinds TE, TF, and TG based on the area of the region EA8 included in the processing result image obtained by the insertion shape element extraction unit 272.

For example, when performing control on the endoscope function control unit 240 in accordance with the control content included in the insertion control information CJE, the insertion control unit 273 acquires auxiliary information HJB in accordance with a backward movement state of the insertion portion 11 by detecting temporal change of the position of the region EA1 included in a processing result image obtained by the insertion shape element extraction unit 272. The above-described auxiliary information HJB can be used to, for example, determine whether it is needed to change the backward movement amount BLA and the backward movement speed BVA included in the insertion control information CJE.

For example, when performing control on the endoscope function control unit 240 in accordance with the control content included in the insertion control information CJG, the insertion control unit 273 acquires auxiliary information HJC in accordance with the backward movement state of the insertion portion 11 by detecting temporal change of the position of the region EA1 included in a processing result image obtained by the insertion shape element extraction unit 272. The above-described auxiliary information HJC can be used to, for example, determine whether it is needed to change the backward movement amount BLB, the backward movement speed BVB, and the rotational angle BAB included in the insertion control information CJG.

For example, when having detected that the regions EA3 and EA8 in a processing result image obtained by the insertion shape element extraction unit 272 have disappeared and regions EA6 and EA7 newly have appeared in the processing result image by performing control on the endoscope function control unit 240 in accordance with the control content included in the insertion control information CJF, the insertion control unit 273 acquires a detection result that the insertion shape of the insertion portion 11 has changed from the kind TF to the kind TG.

For example, when performing control on the endoscope function control unit 240 in accordance with the control content included in the insertion control information CJG, the insertion control unit 273 acquires auxiliary information HJD in accordance with a positional relation between the regions EA6 and EA7 included in the processing result image obtained by the insertion shape element extraction unit 272. The above-described auxiliary information HJD can be used to, for example, determine whether it is needed to change the backward movement amount BLB, the backward movement speed BVB, and the rotational angle BAB included in the insertion control information CJG.

For example, when having detected that the regions EA6 and EA7 in a processing result image obtained by the insertion shape element extraction unit 272 have disappeared by performing control on the endoscope function control unit 240 in accordance with the control content included in the insertion control information CJG, the insertion control unit 273 acquires a detection result that the insertion shape of the insertion portion 11 has changed from the kind TG to the kind TH.

For example, when having detected that none of regions TH2 to TH8 are included in a processing result image obtained by the insertion shape element extraction unit 272 by performing control on the endoscope function control unit 240 in accordance with the control content included in the insertion control information CJF, the insertion control unit 273 acquires a detection result that the insertion shape of the insertion portion 11 has changed from the kind TG to the kind TH.

For example, when no region EA4 has appeared in a processing result image obtained by the insertion shape element extraction unit 272 although control is performed on the endoscope function control unit 240 in accordance with the control content included in the insertion control information CJA, the insertion control unit 273 traces the position of the region EA1 included in the processing result image to acquire a detection result that the insertion shape of the insertion portion 11 has changed from the kind TA to the kind TH.

The present invention is not limited to the above-described embodiments and modification but may include various kinds of changes and applications within the scope of the invention.

For example, the above description is mainly made on a case in which the present invention is an information processing device and an endoscope control device, but the present invention is not limited to the case and may be, for example, an information processing method that performs processing same as processing performed by the information processing device. The present invention may also be an operating method of the endoscope control device.

What is claimed is:

1. An information processing device configured to classify a kind of an insertion shape of an endoscope insertion portion by using information related to the insertion shape of the endoscope insertion portion inserted into a subject, the information processing device comprising a processor including one or more hardware components, wherein the processor is configured to
obtain a classification result that the kind of the insertion shape of the endoscope insertion portion inserted into the subject is classified as one of a plurality of predetermined kinds, and
output the classification result.

2. The information processing device according to claim 1, wherein the processor performs control of an insertion operation of the endoscope insertion portion based on the classification result.

3. The information processing device according to claim 2, wherein the processor performs control based on any of a first operation control group and a second operation control group, the first operation control group being set as control contents for executing alone a basic operation selected from among basic operations of the endoscope insertion portion, the second operation control group being set as control contents for executing a combination of a plurality of basic operations selected from among basic operations achieved by respective functions of an endoscope.

4. The information processing device according to claim 3, wherein the second operation control group is set as control contents for consecutively or simultaneously executing the plurality of basic operations.

5. The information processing device according to claim 2, wherein the processor performs, as control of the insertion operation of the endoscope insertion portion, control of at least one of start, continuation, interrupt, resume, stop, or completion of the insertion operation of the endoscope insertion portion based on the classification result.

6. The information processing device according to claim 2, wherein the processor controls at least one of operation amount, operation speed, or operation force in the insertion operation of the endoscope insertion portion based on the classification result.

7. The information processing device according to claim 2, wherein the processor performs control of the insertion operation of the endoscope insertion portion based on at least one of an image obtained through image pickup of inside of the subject into which the endoscope insertion portion is inserted, information indicating magnitude of external force applied to the endoscope insertion portion, or information indicating the insertion shape of the endoscope insertion portion and based on the classification result.

8. The information processing device according to claim 1, wherein the processor performs processing using a classifier produced by performing machine learning using teacher data including an insertion shape image and a label, the insertion shape image indicating the insertion shape of the endoscope insertion portion, the label indicating a classification result that the insertion shape of the endoscope insertion portion included in the insertion shape image is classified as one of the plurality of predetermined kinds.

9. The information processing device according to claim 1, wherein the processor performs operation for recording the classification result in time series.

10. An endoscope control device configured to perform control of an insertion operation of an endoscope insertion portion by using information related to an insertion shape of the endoscope insertion portion inserted into a subject, the endoscope control device comprising a processor including one or more hardware components, wherein the processor is configured to
obtain an extraction result by extracting one or more constituent elements of the insertion shape of the endoscope insertion portion inserted into the subject, and
perform control of the insertion operation of the endoscope insertion portion based on the extraction result.

11. The endoscope control device according to claim 10, wherein the processor performs control based on any of a first operation control group and a second operation control group, the first operation control group being set as control contents for executing alone a basic operation selected from among basic operations of the endoscope insertion portion, the second operation control group being set as control contents for executing a combination of a plurality of basic operations selected from among basic operations of the endoscope insertion portion.

12. The endoscope control device according to claim 11, wherein the second operation control group is set as control contents for consecutively or simultaneously executing the plurality of basic operations.

13. The endoscope control device according to claim 10, wherein the processor performs, as control of the insertion operation of the endoscope insertion portion, control of at least one of start, continuation, interrupt, resume, stop, or completion of the insertion operation of the endoscope insertion portion based on the extraction result.

14. The endoscope control device according to claim 10, wherein the processor controls at least one of operation amount, operation speed, or operation force in the insertion operation of the endoscope insertion portion based on the extraction result.

15. The endoscope control device according to claim 10, wherein the processor
performs processing for obtaining the extraction result by extracting at least one of an endoscope distal end portion, a loop portion, or an angled portion, and
changes a control content in accordance with temporal change of at least one constituent element included in the extraction result.

16. The endoscope control device according to claim 10, wherein the processor performs control of the insertion operation of the endoscope insertion portion based on at least one of an image obtained through image pickup of inside of the subject into which the endoscope insertion portion is inserted, information indicating magnitude of external force applied to the endoscope insertion portion, or information indicating the insertion shape of the endoscope insertion portion and based on the extraction result.

17. The endoscope control device according to claim 10, wherein the processor performs processing using a classifier produced by performing machine learning using teacher data including an insertion shape image and a label, the insertion shape image indicating the insertion shape of the endoscope insertion portion, the label indicating a classification result that each pixel included in the insertion shape image is classified as one of a plurality of predetermined constituent elements.

18. An information processing method comprising:
obtaining a classification result that a kind of an insertion shape of an endoscope insertion portion inserted into a subject is classified as one of a plurality of predetermined kinds; and
outputting the classification result.

19. The information processing method according to claim 18, further comprising performing control of an insertion operation of the endoscope insertion portion based on the classification result.

20. The information processing method according to claim 18, further comprising performing operation for recording the classification result in time series.

21. An operating method of an endoscope control device configured to perform control of an insertion operation of an endoscope insertion portion by using information related to an insertion shape of the endoscope insertion portion inserted into a subject, the method comprising:
performing processing for obtaining an extraction result by extracting one or more constituent elements of the insertion shape of the endoscope insertion portion inserted into the subject; and
performing control of the insertion operation of the endoscope insertion portion based on the extraction result.
